(19) United States
(12) Patent Application Publication
(10) Pub. No.: US 2019/0275330 A1
(43) Pub. Date: Sep. 12, 2019

(54) Modular Neuromodulation Systems, Methods of Manufacture, and Methods of Treating Rheumatoid Arthritis

(71) Applicant: Galvani Bioelectronics Limited, Brentford, Middlesex (GB)

(72) Inventors: Shivkumar SABESAN, South San Francisco, CA (US); Kedar SHAH, South San Francisco, CA (US)

(21) Appl. No.: 16/349,540

(22) PCT Filed: Nov. 13, 2017
(86) PCT No.: PCT/US2017/061308

§ 371 Date: May 13, 2019

Related U.S. Application Data
(60) Provisional application No. 62/489,064, filed on Apr. 24, 2017.
(60) Provisional application No. 62/421,896, filed on Nov. 14, 2016.

(51) Int. Cl. A61N 1/36; A61N 1/375; A61N 1/372; A61N 1/05; A61B 5/04; A61B 5/00

(52) U.S. Cl. CPC A61N 1/36135; A61N 1/3754; A61N 1/37223; A61B 5/4836; A61B 5/04001; A61N 1/36175; A61N 1/36171; A61N 1/0556

(57) ABSTRACT

The present invention relates to implantable neuromodulation systems and methods, and in particular to modular neuromodulation systems suitable for implantation in a minimally invasive manner, methods of manufacturing the modular neuromodulation systems, and methods of treating rheumatoid arthritis using the modular neuromodulation systems. Particularly, aspects of the present invention are directed to a medical device that includes an implantable neurostimulator including a housing having a width of less than 10 mm and a height of less than 10 mm, one or more feedthroughs that pass through the housing, and an electronics module within the housing and connected to the one or more feedthroughs. The medical device further includes a lead assembly including a lead body including a conductor material, a lead connector that connects the conductor material to the one or more feedthroughs, and one or more electrodes connected to the conductor material.

### CROSS-REFERENCE TO RELATED APPLICATIONS

The present application claims priority and benefit from U.S. Provisional Application No. 62/421,896, filed Nov. 14, 2016, entitled “MODULAR NEUROMODULATION SYSTEMS AND METHODS OF MANUFACTURE” and U.S. Provisional Application No. 62/489,064, filed Apr. 24, 2017, entitled “MODULAR NEUROMODULATION SYSTEMS, METHODS OF MANUFACTURE, AND METHODS OF TREATING RHEUMATOID ARTHRITIS” the entire contents of which are incorporated herein by reference for all purposes.

### FIELD OF THE INVENTION

The present invention relates to implantable neuromodulation systems and methods, and in particular to modular neuromodulation systems suitable for implantation in a minimally invasive manner, methods of manufacturing the modular neuromodulation systems, and methods of treating rheumatoid arthritis using the modular neuromodulation systems.

### BACKGROUND

Normal neural activity is an intricate balance of electrical and chemical signals which can be disrupted by a variety of insults (genetic, chemical or physical trauma) to the nervous system, causing cognitive, motor and sensory impairments. Similar to the way a cardiac pacemaker or defibrillator corrects heartbeat abnormalities, neuromodulation therapies help to reestablish normal neural balance. In particular instances, neuromodulation therapies utilize medical device technologies to enhance or suppress activity of the nervous system for the treatment of disease. These technologies include implantable as well as non-implantable neuromodulation devices and systems that deliver electrical, chemical or other agents to reversibly modify brain and nerve cell activity. The most common neuromodulation therapy is spinal cord stimulation to treat chronic neuropathic pain. In addition to chronic pain relief, some examples of neuromodulation therapies include deep brain stimulation for essential tremor, Parkinson's disease, dystonia, epilepsy and psychiatric disorders such as depression, obsessive compulsive disorder and Tourette syndrome; sacral nerve stimulation for pelvic disorders and incontinence; gastric and colonic stimulation for gastrointestinal disorders such as dysmotility or obesity; vagal nerve stimulation for epilepsy, obesity or depression; carotid artery stimulation for hypertension, and spinal cord stimulation for ischemic disorders such as angina and peripheral vascular disease.

Rheumatoid arthritis is an autoimmune disorder that occurs when the immune system mistakenly attacks body's own tissues. Unlike the wear and tear damage (due to age and/or extreme sports) of osteoarthritis, rheumatoid arthritis affects the lining of the joints, causing a painful swelling that can eventually result in bone erosion and joint deformity. The inflammation associated with rheumatoid arthritis is what can damage other parts of the body as well. While new types of medications have improved treatment options dramatically, severe rheumatoid arthritis can still cause physical disabilities. Recently, neuromodulation has been suggested as a potential treatment option for patients suffering from rheumatoid arthritis. Specifically, electrical stimulation of the splenic plexus has shown promise. However, since patients with rheumatoid arthritis may already be in a pro inflamed state, it is important that device implantation be performed with minimal invasiveness. From this perspective, implantable neuromodulation devices that are designed to be deliverable to an anatomical target such as the splenic plexus using minimally invasive surgery may have a higher probability of reducing the potential for complications in a patient population that is already in an inflamed state.

Conventionally, the neuromodulation devices and systems have a similar form factor or design, derived from their predecessors, the pacemaker or defibrillator. The neuromodulation devices and systems consist of a sizeable implanted pulse generator containing electronics that are connected to leads that deliver electrical pulses to electrodes interfaced with nerves or nerve bundles. The devices are typically expensive, configured for a single therapy, and require invasive surgery to implant the relatively large implanted pulse generator and thread its leads to the appropriate location on the desired nerve or nerve bundle. The surgery requires specialist facilities as well as expert surgeons who can carry out precise lead placement without causing harm. Given the high cost, the need for well-trained surgeons and superior facilities, and the invasiveness of the procedure, today, neuromodulation therapies are offered only as a last resort after all other treatment options have failed, and thus benefit only a small portion of potential patients. Accordingly, the need exists for neuromodulation devices and systems that are relatively inexpensive and can be implanted in a minimally invasive manner while maintaining a soft interface with various nerves and nerve bundles.

### BRIEF SUMMARY

One general aspect includes a medical device including: an implantable neurostimulator including: The medical device also includes a housing having a width of less than 10 mm and a height of less than 10 mm. The medical device also includes a cap bonded to the housing. The medical device also includes one or more feedthroughs that pass through the cap. The medical device also includes an electronics module within the housing and connected to the one or more feedthroughs; and a lead assembly including: a lead body including a conductor material. The medical device also includes a lead connector that connects the conductor material to the one or more feedthroughs; and one or more electrodes connected to the conductor material.

Implementations may include one or more of the following features. The medical device where the implantable neurostimulator further includes a power source within the housing and connected to the electronics module. The medical device where the implantable neurostimulator further includes an antenna connected to the electronics module. The medical device where the antenna is a microwire coil. The medical device where the antenna is within the housing and wrapped around the electronics module. The medical device where the implantable neurostimulator further includes one or more feedthroughs that pass through a proximal end of the implantable neurostimulator, the one or more feedthroughs that pass through the cap are provided at a distal end of the implantable neurostimulator, and the electronics module is connected to the one or more feedthroughs at the proximal end of the implantable neurostimulator. The medical device where the antenna is outside of the housing and connected to the one or more feedthroughs at the proximal end of the implantable neurostimulator. The medical device where the antenna is outside of the housing and connected to the one or more feedthroughs. The medical device where the antenna is a planar coil made of flex substrate and the microwire coil. The medical device where the electronics module includes a pulse generator, a processor, and non-transitory machine readable storage medium having instructions stored thereon that when executed by the processor cause the processor to perform one or more operations. The medical device where the one or more feedthroughs are metal pins that pass through via holes in the cap. The medical device where the one or more feedthroughs include a ferrule that defines an aperture, a conductive element passing through the aperture, and an insulator within the aperture surrounding the conductive element and being brazed to the ferrule. The medical device where the housing is hermetically sealed. The medical device where the housing is ceramic. The medical device where the housing is a metal. The medical device where the housing is cylindrical and the width and height are a diameter of the housing. The medical device where the housing is hemispherical.

Implementations may include one or more of the following features. The medical device where the lead connector is bonding material that directly bonds the conductor material to the one or more feedthroughs. The medical device where the conductor material is bonded via the bonding material to a pin or a bonding pad of the one or more feedthroughs The medical device where the bonding material is conductive epoxy. The medical device where the bonding material is platinum. The medical device where the lead connector is a conductive wire that directly bonds the conductor material to the one or more feedthroughs. The medical device where the lead body includes an insulator and the conductor material, and where the lead connector includes an insulator and a matching conductor material defining a common bore configured to removably receive the lead body such that the conductor material of the lead body is in contact with the matching conductor material of the lead connector. The medical device where the one or more electrodes are connected to the conductor material of the lead body via one or more leads. The medical device where the matching conductor material of the lead connector is connected to the one or more feedthroughs. The medical device where the leady body is a flexible printed circuit or flexible cable The medical device where the lead assembly further includes a flexible cable connected between the lead connector and the one or more feedthroughs. The medical device where the lead body includes an insulator and the conductor material, and where the lead connector includes an insulator and a matching conductor material defining a common bore configured to receive the lead body such that the conductor material of the lead body is in contact with the matching conductor material of the lead connector. The medical device where the one or more electrodes are connected to the conductor material of the lead body via one or more leads. The medical device where the matching conductor material of the lead connector is connected to the flexible cable. The medical device where the one or more electrodes are helical electrodes, probe electrodes, or cuff electrodes.

One general aspect includes a medical device including: an implantable neurostimulator including: The medical device also includes a housing having a width of less than 10 mm and a height of less than 10 mm. The medical device also includes one or more feedthroughs that pass through the housing. The medical device also includes an electronics module within the housing and connected to the one or more feedthroughs; and a lead assembly including: a lead body including: a proximal end having an insulator and a conductor material, and one or more leads sheathed in an insulator and connected to the conductor material; a lead connector including: an insulator and a matching conductor material defining a common bore configured to removably receive the lead body such that the conductor material of the lead body is in contact with the matching conductor material of the lead connector. The medical device also includes one or more electrodes connected to the one or more leads. The medical device also includes where the matching conductor material of the lead connector is connected to the one or more feedthroughs.

Implementations may include one or more of the following features. The medical device where the implantable neurostimulator further includes a power source within the housing and is connected to the electronics module. The medical device where the implantable neurostimulator further includes an antenna connected to the electronics module. The medical device where the antenna is a microwire coil. The medical device where the antenna is within the housing and wrapped around the electronics module. The medical device where the implantable neurostimulator further includes one or more feedthroughs that pass through a proximal end of the implantable neurostimulator, the one or more feedthroughs that pass through the housing are provided at a distal end of the implantable neurostimulator, and the electronics module is connected to the one or more feedthroughs at the proximal end of the implantable neurostimulator. The medical device where the antenna is outside of the housing and connected to the one or more feedthroughs at the proximal end of the implantable neurostimulator. The medical device where the electronics module includes a pulse generator, a processor, and non-transitory machine readable storage medium having instructions stored thereon that when executed by the processor cause the processor to perform one or more operations. The medical device where the one or more feedthroughs are metal pins that pass through via holes in the housing. The medical device where the one or more feedthroughs include a ferrule that defines an aperture, a conductive element passing through the aperture, and an insulator within the aperture surrounding the conductive element and being brazed to the ferrule. The medical device where the housing is hermetically sealed. The medical device where the housing is ceramic. The medical device where the housing is a metal. The medical device where the one or more electrodes are helical electrodes, probe electrodes, or cuff electrodes.

One general aspect includes a medical device including: an implantable neurostimulator including: The medical device also includes a housing that is hemispherical having a width of less than 10 mm and a height of less than 10 mm. The medical device also includes one or more feedthroughs that pass through the housing. The medical device also includes an electronics module within the housing and connected to the one or more feedthroughs; and a lead assembly including: a lead body including: a proximal end having an insulator and a conductor material, and one or more leads sheathed in an insulator and connected to the conductor material; a lead connector including: an insulator and a matching conductor material defining a common bore configured to removably receive the lead body such that the conductor material of the lead body is in contact with the matching conductor material of the lead connector. The medical device also includes one or more electrodes connected to the one or more leads. The medical device also includes where the matching conductor material of the lead connector is connected to the one or more feedthroughs via a flexible cable.

Implementations may include one or more of the following features. The medical device where the implantable neurostimulator further includes a power source within the housing and connected to the electronics module. The medical device where the implantable neurostimulator further includes an antenna connected to the electronics module. The medical device where the antenna is outside of the housing and connected to the one or more feedthroughs. The medical device where the antenna is a planar coil made of flex substrate and a microwire coil. The medical device where the electronics module includes a pulse generator, a processor, and non-transitory machine readable storage medium having instructions stored thereon that when executed by the processor cause the processor to perform one or more operations. The medical device where the one or more feedthroughs are metal pins that pass through via holes in the housing. The medical device where the one or more feedthroughs include a ferrule that defines an aperture, a conductive element passing through the aperture, and an insulator within the aperture surrounding the conductive element and being brazed to the ferrule. The medical device where the housing is hermetically sealed. The medical device where the housing is ceramic. The medical device where the housing is a metal. The medical device where the one or more electrodes are helical electrodes, probe electrodes, or cuff electrodes.

One general aspect includes a method of bonding a flexible printed circuit to a feedthrough, the method including: forming a hole in a substrate of the flexible printed circuit. The method also includes forming a metal annulus on the substrate, where the forming includes patterning the metal annulus around the hole and in contact with a conductive material of the flexible printed circuit. The method also includes placing the feedthrough through the hole. The method also includes joining the feedthrough to the metal annulus.

Implementations may include one or more of the following features. The method where the joining includes: dispensing a conductive epoxy between the feedthrough and the metal annulus; and curing the conductive epoxy. The method where the curing is performed with heat or ultraviolet light. The method further including molding a region surrounding the feedthrough and metal annulus with epoxy or silicone to provide mechanical strain relief and electrical isolation. The method where the joining includes: bending the feedthrough into physical contact with the metal annulus; and attaching the feedthrough to the metal annulus such that the feedthrough is in electrical contact with the metal annulus. The method where the attaching includes: dispensing a conductive epoxy between the feedthrough and the metal annulus; and curing the conductive epoxy. The method where the attaching includes welding, thermo-compression bonding, or ultrasonic bonding of the feedthrough to the metal annulus. The method further including molding a region surrounding the feedthrough and the metal annulus with epoxy or silicone to provide mechanical strain relief and electrical isolation. The method where the mechanical strain relief is achieved using a tapered geometry that gradually decreases from a width and height of a neurostimulator to a width and height of the flexible printed circuit. The method where the feedthrough traverses a housing or cap of the neurostimulator.

One general aspect includes a method of bonding a flexible printed circuit to a feedthrough, the method including: forming a bond pad on a substrate of the flexible printed circuit, where the forming includes patterning the bond pad in contact with a conductive material of the flexible printed circuit. The method also includes joining the feedthrough to the bond pad.

Implementations may include one or more of the following features. The method where the joining includes: bending the feedthrough into physical contact with the bond pad; and attaching the feedthrough to the bond pad such that the feedthrough is in electrical contact with the bond pad. The method where the attaching includes: dispensing a conductive epoxy between the feedthrough and the bond pad; and curing the conductive epoxy. The method where the attaching includes welding, thermo-compression bonding, or ultrasonic bonding of the feedthrough to the bond pad. The method further including molding a region surrounding the feedthrough and the bond pad with epoxy or silicone to provide mechanical strain relief and electrical isolation. The method where the method further includes trimming the feedthrough such that the feedthrough protrudes to a height of <2 mm from an outer planar surface of a feedthrough assembly. The method further including affixing a backer to the substrate. The method where the joining includes welding, thermo-compression bonding, or ultrasonic bonding of the feedthrough to the bond pad, and where the backer is on a first side of the substrate opposite to that of the outer planar surface of the feedthrough and the bond pad is on a second side of the substrate adjacent to the outer planar surface of the feedthrough. The method further including molding a region surrounding the feedthrough and the bond pad with epoxy or silicone to provide mechanical strain relief and electrical isolation. The method where the joining includes bonding a first end of a wire to the feedthrough and bonding a second end of the wire to the bond pad. The method further including molding a region surrounding the feedthrough and the bond pad with epoxy or silicone to provide mechanical strain relief and electrical isolation. The method where the joining includes bonding a first end of a metal tab to the feedthrough and bonding a second end of the metal tab to the bond pad. The method further including molding a region surrounding the feedthrough and the bond pad with epoxy or silicone to provide mechanical strain relief and electrical isolation. The method where the mechanical strain relief is achieved using a tapered geometry that gradually decreases from a width and height of a neurostimulator to a width and height of the flexible printed circuit The method where the bond bad is patterned near an edge of the substrate. The method where the feedthrough traverses a housing of the neurostimulator.

One general aspect includes a method of implanting a medical device, the method including: obtaining a trocar having a diameter of less than 20.0 mm; using the trocar to gain access to a body cavity including an implantation site for the medical device; feeding the medical device through the trocar to the implantation site, where the medical device includes: (i) a neurostimulator including a housing having a width of less than 10 mm and a height of less than 10 mm, and (ii) a lead assembly including one or more electrodes connected to the neurostimulator. The method also includes placing the one or more electrodes into contact with a nerve or artery/nerve plexus at the implantation site such that a neural interface is created between the one or more electrodes and the nerve or artery/nerve plexus. The method also includes placing the neurotransmitter subdurally at a location that is either remote from or at the neural interface.

Implementations may include one or more of the following features. The method where the nerve or artery/nerve plexus includes peripheral nerves near a splenic artery or a splenic artery/nerve plexus. The method the trocar has a diameter of less than 12 mm. The method where the one or more electrodes are helical electrodes, probe electrodes, or cuff electrodes. The method further including obtaining another trocar and an optics system, using the another trocar to gain access to the body cavity, feeding the optics system through the another trocar to the implantation site, and obtaining images of the medical device at the implantation site.

One general aspect includes a method of treating an inflammatory related disease including: implanting a medical device in a body cavity using a laparoscopic procedure, where the medical device includes: (i) a neurostimulator including a housing having a width of less than 10 mm and a height of less than 10 mm, and (ii) a lead assembly including one or more electrodes connected to the neurostimulator, and the implanting includes connecting the one or more electrodes to a nerve or artery/nerve plexus in the body cavity. The method also includes delivering, by a computing system, neural stimulation to the nerve or artery/nerve plexus based on a first set of stimulation parameters. The method also includes monitoring, by the computing system, a response to the neural stimulation that includes monitoring responses of the nerve or artery/nerve plexus and a physiological parameter change. The method also includes modifying, by the computing system, the first set of the stimulation parameters based on the responses of the nerve or artery/nerve plexus and the physiological parameter change to create a second set of stimulation parameters. The method also includes delivering, by the computing system, the neural stimulation based on the second set of the stimulation parameters. Other embodiments of this aspect include corresponding computer systems, apparatus, and computer programs recorded on one or more computer storage devices, each configured to perform the actions of the methods.

Implementations may include one or more of the following features. The method where the stimulation parameters include at least one of: stimulation amplitude, pulse width, frequency, duty cycle, stimulation waveform shape, and electrode configuration. The method where the physiological parameter change includes a change in inflammation of a patient. The method where the monitoring the response to the neural stimulation includes determining whether the neural stimulation has a desired physiological effect on the inflammation of the patient. The method where the determining whether the neural stimulation has an effect on the inflammation of the patient includes obtaining values for biomarkers of the inflammation, comparing the values for the biomarkers to baseline values to determine an extent of change in the inflammation, and comparing the extent of change in the inflammation to one or more predetermined thresholds to determine whether the neural stimulation provided using the first set of stimulation parameters achieved the desired physiological effect, had no physiological effect, or an adverse physiological effect. The method where when the neural stimulation has the adverse physiological effect, modifying the first set of the stimulation parameters based on the responses of the nerve or artery/nerve plexus and the physiological parameter change to create the second set of stimulation parameters. The method where the method further includes modifying the first set of the stimulation parameters based on a titration schedule and the physiological parameter change to create the second set of stimulation parameters. The method where the method further includes determining whether adequate adaptation is achieved. The method where the adequate adaptation is achieved when at least one of the following objectives is achieved: a target intensity level for one or more of the stimulation parameters and/or a desired physiological effect. The method where when the adequate adaptation is not achieved, modifying the first set of the stimulation parameters based on the titration schedule and the physiological parameter change to create the second set of stimulation parameters. The method where the nerve or artery/nerve plexus includes peripheral nerves near a splenic artery or a splenic artery/nerve plexus. Implementations of the described techniques may include hardware, a method or process, or computer software on a computer-accessible medium.

### BRIEF DESCRIPTION OF THE DRAWINGS

The present invention will be better understood in view of the following non-limiting figures, in which:

FIGS. 1A, 1B, 1C, 1D, 1E, and 1F show an implantable neurostimulator or IPG in accordance with some aspects of the present invention;

FIGS. 2A and 2B show a medical device (e.g., a module neuromodulation device or system) comprising an implantable neurostimulator or IPG and a lead assembly in accordance with some aspects of the present invention;

FIGS. 3A, 3B, 3C, 3D, and 3E show an implantable neurostimulator or IPG in accordance with some alternative aspects of the present invention;

FIGS. 4A, 4B, 4C and 4D show a medical device (e.g., a module neuromodulation device or system) comprising an implantable neurostimulator or IPG and a lead assembly in accordance with some alternative aspects of the present invention;

FIGS. 5-8 show exemplary flows for bonding a flexible printed circuit to a feedthrough in accordance with some aspects of the present invention;

FIG. 9 shows a front anatomical diagram illustrating placement of a neuromodulation device or system in a patient in accordance with some aspects of the present invention; and

FIGS. 10-12 show exemplary flows for implanting neuromodulation devices or systems and treating inflammatory related diseases in accordance with some aspects of the present invention.

### DETAILED DESCRIPTION

I. Introduction

In various embodiments, the present invention is directed to a neuromodulation device or system including an implantable neurostimulator (e.g., an implanted pulse generator (IPG)) and a lead assembly having one or more electrodes (e.g., a neural stimulator). A problem associated with conventional neuromodulation devices and systems, however, is that they are typically designed for a single neuromodulation therapy (i.e., the components lack modularity), and the IPG is bulky and implanted via an open surgical exposure to a target location remote from the electrodes. These approaches are both inefficient from a cost standpoint for the number of neuromodulation therapies that are projected to exist in the future and unreasonably invasive and complex to replace traditional forms of treatment such as pharmaceuticals.

To address these problems, the present invention is directed to neuromodulation devices or systems that have the modularity of traditional pacemakers where neural interfaces and the IPG are inter-changeable, while achieving a less complex and minimally invasive implantation. For example, one illustrative embodiment of the present disclosure comprises: (i) an implantable neurostimulator including a housing having a width of less than 10 mm and a height of less than 10 mm, one or more feedthroughs that pass through the housing, and an electronics module within the housing and connected to the one or more feedthroughs, and (ii) a lead assembly including a lead body including a conductor material, a lead connector that connects the conductor material to the one or more feedthroughs, and one or more electrodes connected to the conductor material. In some aspects, the lead body includes an insulator and the conductor material, and the lead connector includes an insulator and a matching conductor material defining a common bore configured to removably receive the lead body such that the conductor material of the lead body is in contact with the matching conductor material of the lead connector. In additional or alternative embodiments, the lead body is a flexible printed circuit and aspects of the present invention are directed to methods of connecting the flexible printed circuit to the one or more feedthroughs.

Advantageously, these approaches provide neuromodulation devices and systems that are relatively inexpensive and can be implanted in a minimally invasive manner while maintaining a soft interface with various nerves and nerve bundles. For example, all of the components of the neuromodulation devices and systems including the housing, the electronics module, the feedthroughs, and the lead assembly are capable of being independently manufactured and used in different systems to achieve modularity in design. Further, the lead connector allows for different electrodes (e.g., book electrodes, cuff electrodes, spiral cuff electrodes, epidural electrodes, helical electrodes, probe electrodes, linear electrodes, paddle electrodes, intraneural electrodes, etc.) to be interfaced with different implantable neurostimulators or IPGs inside or outside of the patient's body. Moreover, the sizing of the housing (e.g., a housing having a width of less than 10 mm and a height of less than 10 mm) of the implantable neurostimulators or IPGs is maintained small enough such that they can be implanted in a less complex and minimally invasive manner, for example, through a trocar or cannula.

II. Neuromodulation Devices or Systems

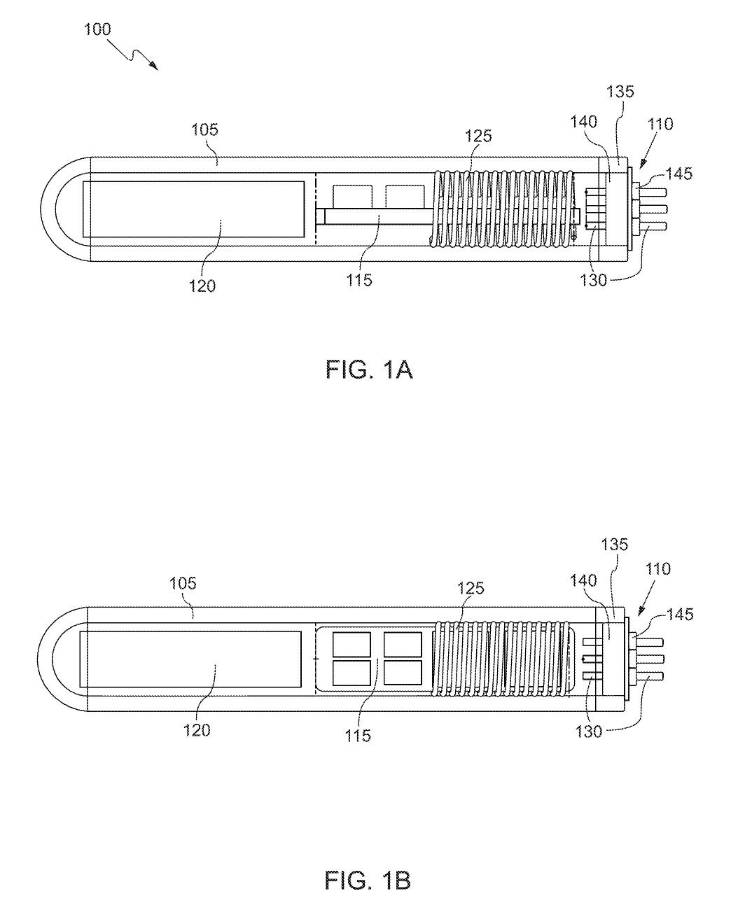

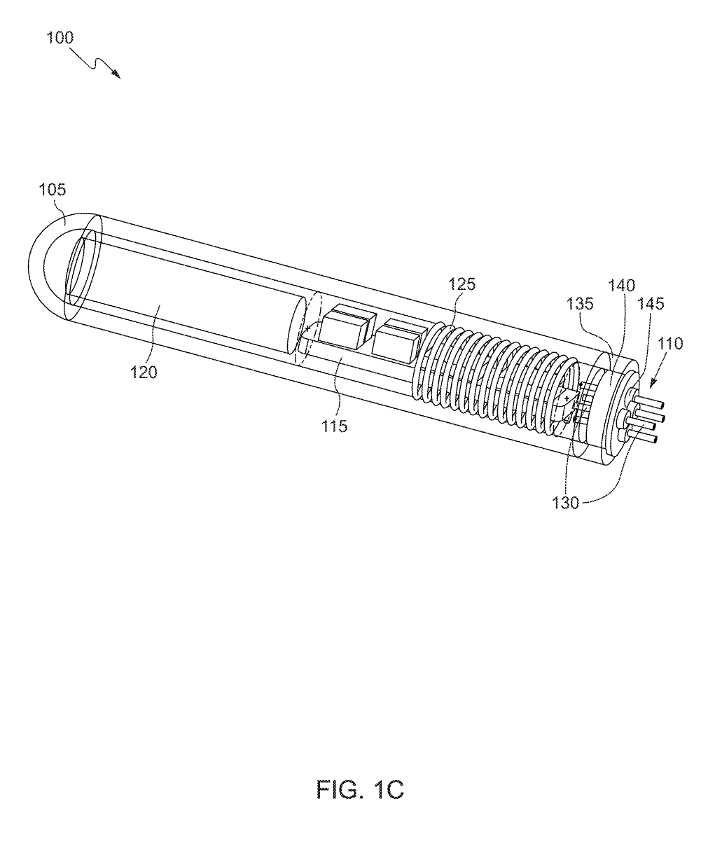

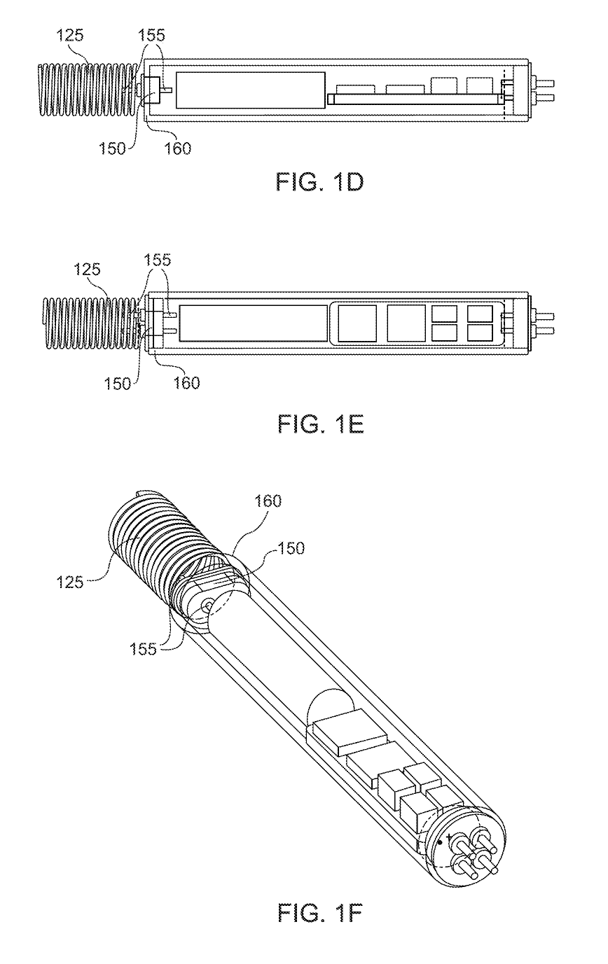

FIGS. 1A, 1B, and 1C show as implantable neurostimulator or IPG 100 in accordance with some aspects of the present invention. In some embodiments, the neurostimulator 100 includes a housing 105, a feedthrough assembly 110, an electronics module 115, a power source 120, and an antenna 125. The housing 105 may be comprised of materials that are biocompatible such as bioceramics or bioglasses for radio frequency transparency, or metals such as titanium. The size and shape of the housing 105 are selected such that the neurostimulator 100 can be implanted in a less complex and minimally invasive manner, for example, through a trocar or cannula. As such, in various embodiments, the housing 105 has a width of less than 10 mm, for example from 2 mm to 6 mm, a height of less than 10 mm, for example from 2 mm to 6 mm, a length of less than 70 mm, for example from 20 mm to 40 mm, and a cross-sectional area of less than 150 mm², for example from 75 mm² to 120 mm². In some embodiments, the housing 105 may have a pill-shaped cylindrical form and the width and height are a diameter of the housing as shown in FIGS. 1A, 1B, and 1C.

The feedthrough assembly 110 is attached to a hole in distal end of the housing 105 and is attached so that the housing 105 is hermetically sealed. The feedthrough assembly 110 can include one or more feedthroughs 130 (i.e., electrically conductive elements, pins, wires, tabs, pads, etc.) mounted within and extending through an end of the housing 105 or a cap 135 from an interior of the housing 105 to an exterior of the housing 105. In certain examples, the one or more feedthroughs 130 are made of metal such as copper, silver, or gold. The cap may be formed of bioceramics, bioglasses, or metals such as titanium. In embodiments that include the cap 135, the cap 135 may be mounted to the housing 105 of the neurostimulator 100 by fitting the cap 135 into a hole in the housing 105 and metallic (e.g., gold) brazing, diffusion bonding, or laser welding the cap 135 at an outer perimeter of the cap 135.

The feedthrough assembly 110 includes a metallic ferrule 140 that defines an aperture. In an example, the ferrule 140 is mounted to the housing 105 or the cap 135 of the neurostimulator 100 by fitting the ferrule 140 into a hole in the housing 105 and metallic (e.g., gold) brazing or laser welding the ferrule 140 at an outer perimeter of the ferrule 140. In certain embodiments, the ferrule 140 is formed of titanium. An insulator 145 is mounted within the aperture of the ferrule 140. In certain examples, the insulator 145 includes a bioceramic material, such as an alumina or zirconia ceramic. The insulator 145 can be mounted to the ferrule 140 using metallic brazing, for example. The insulator 145 includes one or more via holes extending through the insulator 145 (i.e., through the aperture of the ferrule 140). The one or more feedthroughs 130 are mounted within and extend through the respective via holes of the insulator 145 from an interior side of the feedthrough assembly 110 to an exterior side of the feedthrough assembly 110. The feedthroughs 130 are hermetically connected to the insulator 145 at the via holes using a metallic-brazed joint or co-fired joint, for example.

With the feedthroughs 130 in sealing engagement with housing 105, interior ends of the feedthroughs 130 project from the interior surface of the housing 105 or cap 135 into the interior of the housing 105 and may be terminated with a termination pad. In certain embodiments, the termination pad generally lies perpendicular to the longitudinal axis extending through the feedthroughs 130. Exterior ends of the feedthroughs 130 project from the exterior surface of the housing 105 or cap 135 to the exterior of the housing 105. Each of the exterior ends extends for connection to a corresponding conductor of a lead. Each of the interior ends extends for connection to the electronics module 115 also located within the interior of housing 105.

The electronics module 115 is connected (e.g., electrically connected) to the interior ends of the one or more feedthroughs 130 such that the electronics module 115 is able to apply a signal or electrical current to each of the leads connected to the exterior ends of the feedthroughs 130. In some embodiments, the electronics module 115 is connected to the interior ends of the one or more feedthroughs 130 via indirect connection such as soldered wires or tabs, or direct connection via solder, laser welding, crimping, etc. The electronics module 115 includes any discrete and/or integrated electronic circuit components that implement analog and/or digital circuits capable of producing the functions attributed to the neuromodulation devices or systems described herein. The electronic circuits components are assemble to form the electronics module 115 using various combinations of solder reflow, wire-bonding, flip-chip bonding, etc.

In various embodiments, the electronics module 115 may include electronic circuit components such as a pulse generator that generates a signal or electrical current and one or more processors that determine or sense electrical activity via electrodes and/or deliver electrical stimulation via electrodes. Additionally, electronics module 115 may also include non-transitory machine readable storage medium having instructions stored thereon that when executed by the processor cause the processor to perform one or more operations such as generation of a signal or electric current. Electronics module 115 may also include sensors that sense physiological conditions of a patient, such as an accelerometer and/or a pressure sensor. In certain embodiments, the electronics module 115 is a printed circuit board with an interposer in combination with discrete and/or integrated electronic circuit components such as application specific integrated circuits (ASICs) assembled using either 2.5 or 3D integration to achieve miniaturization.

The power source 120 is within the housing 105 and connected (e.g., electrically connected) to the electronics module 115 to power and operate the components of the electronics module 115. In some embodiments, the power source 120 is connected to the electronics module 115 via indirect connection such as soldered or welded wires or tabs, or direct connection via solder, laser welding, crimping, etc. The power source 120 may be any type of device that is of implant grade and configured to hold a charge to power and operate the components of the electronics module 115. In certain embodiments, the power source is a non-rechargeable battery needing replacement every few years (depending on stimulation parameters) or a rechargeable battery that is replenished via an external inductive charging system.

The antenna 125 is connected (e.g., electrically connected) to the electronics module 115 for wireless communication with external devices via radiofrequency (RF) telemetry. In some embodiments, the antenna 125 is connected to the electronics module 115 via indirect connection such as soldered or welded wires or tabs, or direct connection via solder, laser welding, crimping, etc. The wireless communication implemented via the antenna 125 may include receiving information or signals such as power on/off signals, configuration packages to update software, software setting data to configure software, physiological data such a blood pressure from implantable or external sensors, etc., and relay important information or signals (e.g., electrocardiogram and blood pressure) from sensors or the one or more processors on the electronics module 115 or the electrodes to external equipment to be analyzed or to guide treatment.

In some embodiments, such as when the housing 105 is made of bioceramics or bioglass for radio frequency transparency, the antenna 125 is housed within the housing 105 (as shown in FIGS. 1A, 1B, and 1C). For example, the antenna 125 may be a microwire in various shapes and locations within the interior of housing 105, e.g., a coil wrapped around the electronics module 115 or disposed on an internal surface of the housing 105. The microwire may be made of a metal such as copper, silver, or gold. In alternative embodiments, such as when in the housing 105 is made of metal or a material that blocks radio frequency transmissions, the antenna 125 is outside the housing 105 (as shown in FIGS. 1D, 1E, and 1F). For example, the antenna 125 may be a microwire in various shapes and locations exterior to the housing 105, e.g., a coil located at a proximal end of the housing 105 or disposed on an external surface of the housing 105.

In embodiments in which the antenna 125 is external to the housing (as shown in FIGS. 1D, 1E, and 1F), the antenna 125 may be connected to (e.g., electrically connected) the electronics module 115 via the feedthrough assembly 110 located at the distal end of the housing 105 or an additional feedthrough assembly 150 located at a proximal end of the housing 105. The feedthrough assembly 150 is attached to a hole in proximal end of the housing 105 and is attached so that the housing 105 is hermetically sealed. The feedthrough assembly 150 can include one or more feedthroughs 155 (i.e., electrically conductive pins or feedthroughs) mounted within and extending through an end of the housing 105 or a cap 160 from an interior of the housing 105 to an exterior of the housing 105, as similarly described with respect to the feedthrough assembly 110.

With the feedthroughs 155 in sealing engagement with housing 105, interior ends of the feedthroughs 155 project from the interior surface of the housing 105 or cap 160 into the interior of the housing 105 and may be terminated with a termination pad. In certain embodiments, the termination pad generally lies perpendicular to the longitudinal axis extending through the feedthroughs 155. Exterior ends of the feedthroughs 155 project from the exterior surface of the housing 105 or cap 160 to the exterior of the housing 105. Each of the exterior ends extends for connection to a corresponding conductor of the antenna 125. Each of the interior ends extends for connection to the electronics module 115 located within the interior of housing 105.

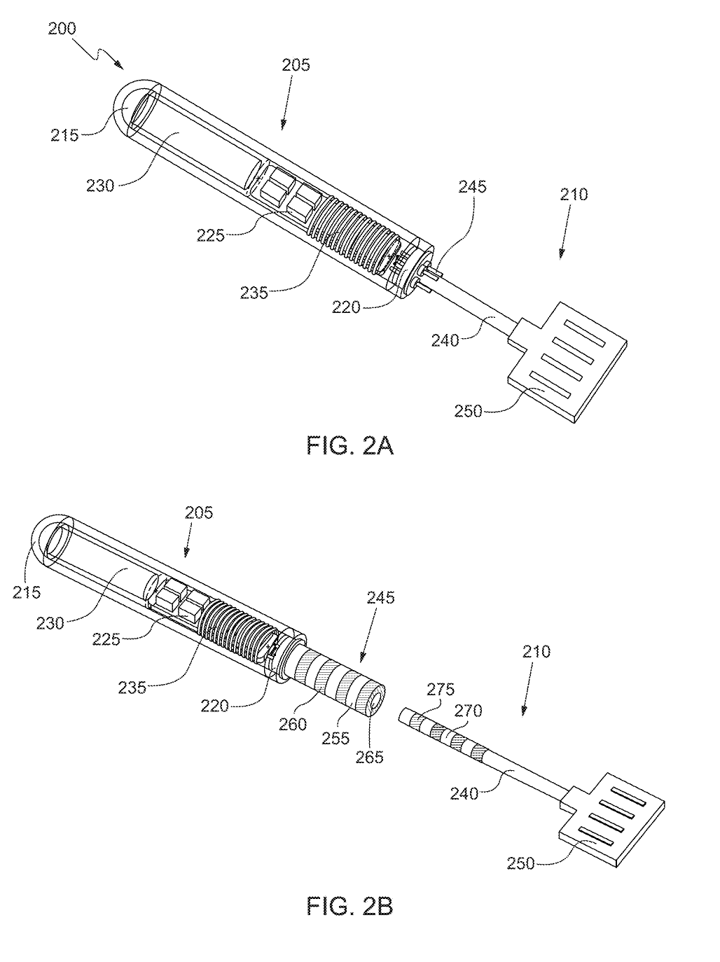

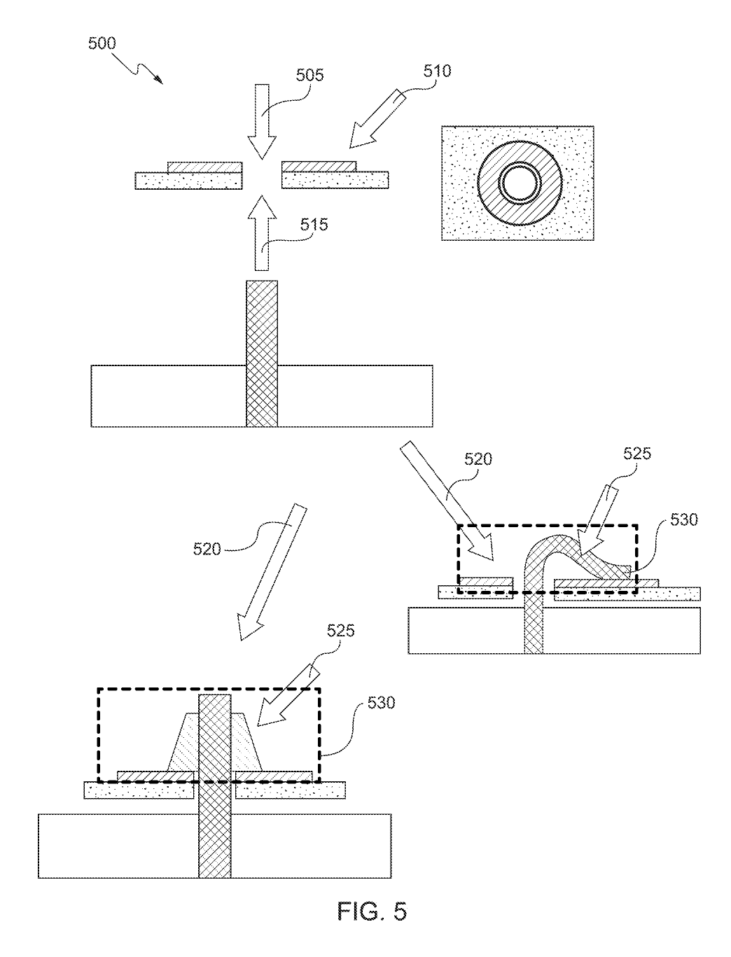

FIGS. 2A and 2B show a medical device 200 (e.g., a module neuromodulation device or system) comprising an implantable neurostimulator or IPG 205 (e.g., the neurostimulator 100 described with respect to FIGS. 1A-1F) and a lead assembly 210 in accordance with some aspects of the present invention. In some embodiments, the neurostimulator 205 includes a housing 215, a feedthrough assembly 220, an electronics module 225, a power source 230, and an antenna 235, as similarly described with respect to FIGS. 1A-1F. The lead assembly 210 may include a lead body 240, a lead connector 245, and one or more electrodes 250. In some embodiments, the lead connector 245 is bonding material that directly bonds conductor material of the lead body 240 to the one or more feedthroughs of the feedthrough assembly 220 (see, e.g., FIG. 2A). The bonding material may be a conductive epoxy or a metallic solder or weld such as platinum. In other embodiments, the lead connector 245 is conductive wire or tab (e.g., a wire or tab formed of copper, silver, or gold) that directly bonds conductor material of the lead body 240 to the one or more feedthroughs of the feedthrough assembly 220. In yet other embodiments, the lead connector 245 is a point of contact or contact region encased in a non-conductive epoxy or an insulator that directly bonds conductor material of the lead body 240 to the one or more feedthroughs of the feedthrough assembly 220. The housing 105 or cap 135 and the lead body 240 may be designed to connect with one another (e.g., via a pin and sleeve connector, snap and lock connector, flexible printed circuit connectors, or manufacturing methods as described herein with respect to FIGS. 5-8). Further, the lead connector 245 and the lead body 240 may be overmolded with non-conductive epoxy or silicone to provide mechanical strain relief and electrical isolation. The mechanical strain relief may provide protection from inadvertent bending during implantation, or for bending while implanted in the patient. Further, the strain relief may also provide protection from tension on the lead connector 245 or on the neural interface including the leady body 240 and one or more electrodes 250.

In alternative embodiments, the lead connector 245 is an integrated seal and electrical contact system including regions of insulator material 255 and regions of conductor material 260 (see, e.g., FIG. 2B) that define a common bore 265 configured to removably receive the lead body 240 such that the conductor material of the lead body 240 is in contact with the matching conductor material 260 of the lead connector 245. The conductor material 260 is connected to each respective feedthrough of the feedthrough assembly 220 via a conductive wire, pad, or tab. The insulator material 255 may be silicone and have a diameter of at least 2 mm, for example from 3 mm to 7 mm. The conductor material 260 may be formed of a conductive metal such as a copper, silver, gold, platinum, stainless steel, nickel-cobalt base alloy, platinum-iridium alloy, brass, bronze, aluminum, etc., and have a diameter of at least 2 mm, for example from 3 mm to 7 mm. The common bore 265 should be configured to removably receive the lead body 240 based on the dimensions and material of the insulator material 255 and the conductor material 260 such that a sliding insertion force of the lead body 240 is less than 1.5 N, for example from 0.6 N to 1.2 N, and an extraction force of the lead body 240 is less than 2.1 N, for example from 1.7 N to 1.2 N.

The lead body 240 includes one or more leads of conductive material and insulator. The one or more leads carry electrical conductors that allow electrical coupling of the electronics module 225 to the one or more electrodes 250 via the lead connector 245 and feedthrough assembly 220. In some examples the one or more leads are extruded with a dielectric material such as a polymer having suitable dielectric, flexibility and biocompatibility characteristics. Polyurethane, polycarbonate, silicone, polyethylene, fluoropolymer and/or other medical polymers, copolymers and combinations or blends can be used. In some embodiments, the conductive material for the one or more leads may serve as a strengthening member onto which the body of the lead is extruded. For example, a distal electrode may couple to a centrally located wire on which the body of lead is extruded. The conductive material may be any suitable conductor such as stainless steel, silver, copper or other conductive materials, which may have separate coatings or sheathing for anticorrosive, insulative and/or protective reasons. The conductive material may take various forms including wires, drawn filled tubes, helical coiled conductors, microwires, and/or printed circuits, for example. The lead body 240 may take various forms including a flexible printed circuit or a flexible cable, for example.

In embodiments in which the lead connector 245 is an integrated seal and electrical contact system, the lead body 240 includes regions of insulator material 270 and regions of conductor material 275 (see, e.g., FIG. 2B) at a proximal end that define a stacked rigid region configured to be removably inserted into the bore 265 of the lead connector 245 such that the conductor material 275 is in contact with the matching conductor material 260 of the lead connector 245. The contact between the conductor material 275 and the matching conductor material 260 allows for the conductive material of the one or more leads in the lead body 240 to be in electrical contact with the conductor material 260, and thus allows for electrical coupling of the electronics module 225 to the one or more electrodes 250 via the lead connector 245 and feedthrough assembly 220. The conductor material 275 is connected to each respective lead of the lead body 240 via a conductive wire, pad, or tab. The insulator material 270 may be silicone and have a diameter of at least 1 mm, for example from 1.2 mm to 6 mm. The conductor material 275 may be formed of a conductive metal such as a copper, silver, gold, platinum, stainless steel, nickel-cobalt base alloy, platinum-iridium alloy, brass, bronze, aluminum, etc., and have a diameter of at least 1 mm, for example from 1.2 mm to 6 mm.

The one or more electrodes 250 are connected to the conductor material of the lead body 240 via the one or more leads. In some embodiments, the one or more electrodes 250 are placed around, within or adjacent to a nerve trunk or root to stimulate the nerve and/or sense electrical impulses traveling through the nerve. The one or more electrodes 250 may be formed of a conductive material such as a copper, silver, gold, platinum, stainless steel, nickel-cobalt base alloy, platinum-iridium alloy, brass, bronze, aluminum, etc., and take the form of book electrodes, cuff electrodes, spiral cuff electrodes, epidural electrodes, helical electrodes, probe electrodes, linear electrodes, paddle electrodes, and intraneural electrodes, for example.

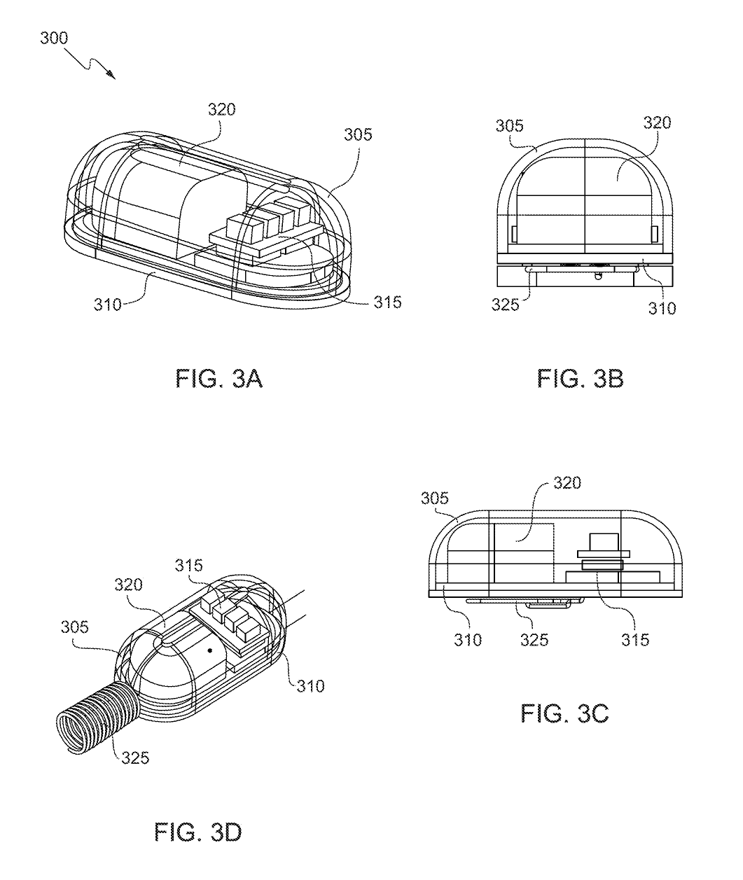

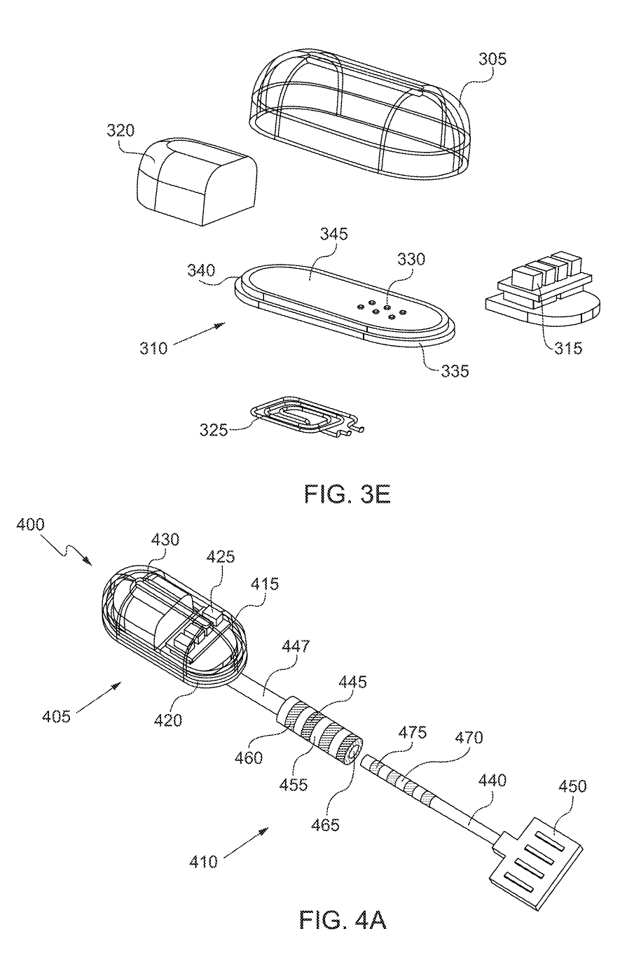

FIGS. 3A, 3B, 3C, 3D, and 3E show as implantable neurostimulator or IPG 300 in accordance with some aspects of the present invention. In some embodiments, the neurostimulator 300 includes a housing 305, a feedthrough assembly 310, an electronics module 315, a power source 320, and an antenna 325. The housing 305, feedthrough assembly 310, electronics module 315, power source 320, and antenna 325 are similarly designed and constructed as the housing 105, feedthrough assembly 110, electronics module 115, power source 120, and antenna 125 as described with respect to FIGS. 1A-1F, and thus the descriptions thereof are not repeated here but for variations in the design and construction. For example, the housing 305 has a width of less than 10 mm, for example from 2 mm to 6 mm, a height of less than 15 mm, for example from 4 mm to 10 mm, a length of less than 70 mm, for example from 20 mm to 40 mm, and a cross-sectional area of less than 200 mm², for example from 90 mm² to 150 mm². In some embodiments, the housing 305 may have an elongated hemispherical shape as shown in FIGS. 3A, 3B, 3C, 3D, and 3E.

The feedthrough assembly 310 is attached to a bottom of the housing 305 and is attached so that the housing 305 is hermetically sealed. The feedthrough assembly 310 can include one or more feedthroughs 330 (i.e., electrically conductive pins, wires, tabs, pads, etc.) mounted within and extending through an end of the housing 305 or a cap 335 from an interior of the housing 305 to an exterior of the housing 305. In embodiments that include the cap 335, the cap 335 may be mounted to the housing 305 of the neurostimulator 300 by fitting the cap 335 into the bottom in the housing 305 and metallic (e.g., gold) brazing, diffusion bonding, or laser welding the cap 335 at an outer perimeter of the cap 335. In some embodiments, the cap 335 is machined from biocompatible material such as titanium or nickel, and laser welded at an outer perimeter of the cap 335. In alternative embodiments, the cap 335 is formed from biocompatible material such as bioceramics or bioglasses, and diffusion bonded to a metal ferrule or the housing 305.

The feedthrough assembly 310 includes a planar metallic ferrule 340. In an example, the ferrule 340 is mounted to the housing 305 or the cap 335 of the neurostimulator 300 by fitting the ferrule 340 into the bottom of the housing 305 and metallic (e.g., gold) brazing or laser welding the ferrule 340 at an outer perimeter of the ferrule 340. A planar substrate 345, e.g., an insulator, is mounted within the ferrule 340. In certain examples, the substrate 345 includes a bioceramic material, such as an alumina or zirconia ceramic. The substrate 345 can be mounted to the ferrule 340 using metallic brazing, for example. The substrate 345 includes one or more via holes extending through the substrate 345. The one or more feedthroughs 330 are mounted within and extend through the respective via holes of the insulator 345 from an interior side of the feedthrough assembly 310 to an exterior side of the feedthrough assembly 310. The feedthroughs 330 are hermetically connected to the substrate 345 at the via holes using a metallic-brazed joint or co-fired joint, for example.

The antenna 325 is connected (e.g., electrically connected) to the electronics module 315 for wireless communication with external devices via radiofrequency (RF) telemetry. In some embodiments, the antenna 325 is connected to the electronics module 315 via indirect connection such as soldered or welded wires or tabs, or direct connection via solder, laser welding, crimping, etc. In some embodiments, such as when the housing 305 is made of bioceramics or bioglass for radio frequency transparency, the antenna 325 is housed within the housing 305. For example, the antenna 325 may be a microwire or planar wire made of flex substrate in various shapes and locations within the interior of housing 305, e.g., disposed on an internal surface of the substrate 345 or inn surface of the housing 305. The microwire or planar wire may be made of a metal such as copper, silver, or gold.

In alternative embodiments, such as when in the housing 305 is made of metal or a material that blocks radio frequency transmissions, the antenna 325 is outside the housing 305 (as shown in FIGS. 3B, 3C, 3D, and 3E). For example, the antenna 125 may be a microwire or planar wire in various shapes and locations exterior to the housing 105, e.g., disposed on an external surface of the substrate 345, a coil located at a proximal end of the housing 305, or disposed on an external surface of the housing 305. In embodiments in which the antenna 325 is external to the housing (as shown in FIGS. 3B, 3C, 3D, and 3E), the antenna 325 may be connected to (e.g., electrically connected) the electronics module 315 via the feedthrough assembly 310 located at the bottom of the housing 305.

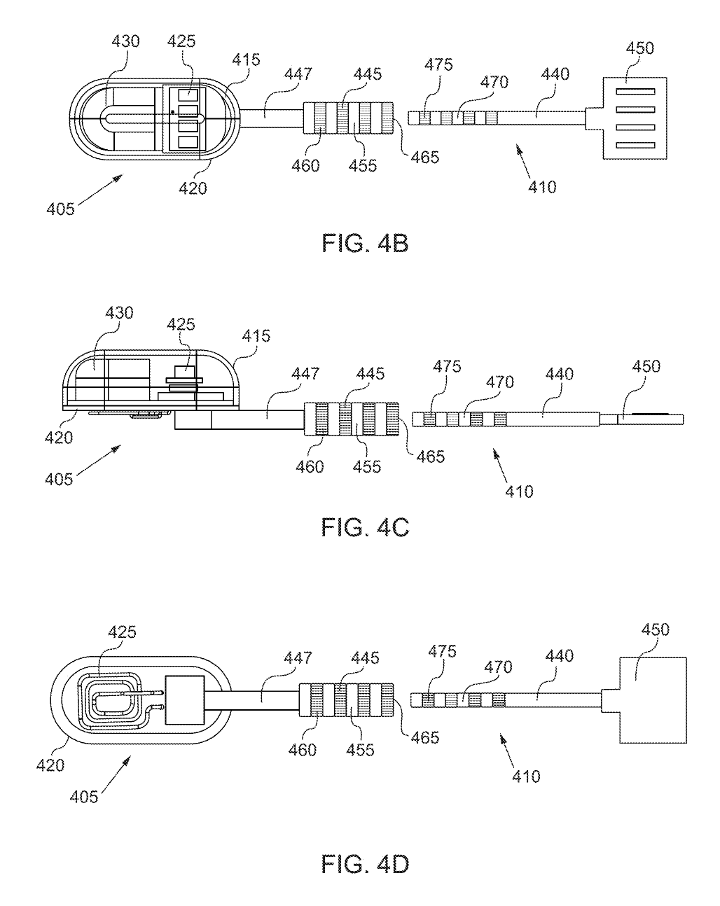

FIGS. 4A, 4B, 4C, and 4D show a medical device 400 (e.g., a module neuromodulation device or system) comprising an implantable neurostimulator or IPG 405 (e.g., the neurostimulator 300 described with respect to FIGS. 3A-3E) and a lead assembly 410 in accordance with some aspects of the present invention. In some embodiments, the neurostimulator 405 includes a housing 415, a feedthrough assembly 420, an electronics module 425, a power source 430, and an antenna 435, as similarly described with respect to FIGS. 3A-3E. The lead assembly 410 may include a lead body 440, a lead connector 445, a flexible cable 447, and one or more electrodes 450. The lead body 440, lead connector 445, and one or more electrodes 450 are similarly designed and constructed as the lead body 240, lead connector 245, and one or more electrodes 250 as described with respect to FIGS. 2A and 2B, and thus the descriptions thereof are not repeated here but for variations in the design and construction

In some embodiments, the lead connector 345 is an integrated seal and electrical contact system including regions of insulator material 355 and regions of conductor material 360 (see, e.g., FIG. 4A) that define a common bore 465 configured to removably receive the lead body 440 such that the conductor material of the lead body 240 is in contact with the matching conductor material 460 of the lead connector 445. The conductor material 460 is connected to each respective feedthrough of the feedthrough assembly 420 via the flexible cable 447. The insulator material 455 may be silicone and have a diameter of at least 2 mm, for example from 3 mm to 7 mm. The conductor material 460 may be formed of a conductive metal such as a copper, silver, gold, platinum, stainless steel, nickel-cobalt base alloy, platinum-iridium alloy, brass, bronze, aluminum, etc., and have a diameter of at least 2 mm, for example from 3 mm to 7 mm. The common bore 465 should be configured to removably receive the lead body 440 based on the dimensions and material of the insulator material 455 and the conductor material 460 such that a sliding insertion force of the lead body 440 is less than 1.5 N, for example from 0.6 N to 1.2 N, and an extraction force of the lead body 440 is less than 2.1 N, for example from 1.7 N to 1.2 N.

The lead body 440 includes one or more leads of conductive material and insulator. The one or more leads carry electrical conductors that allow electrical coupling of the electronics module 425 to the one or more electrodes 450 via the lead connector 445, flexible cable 447, and feedthrough assembly 420. In some embodiments, the lead connector 445 is an integrated seal and electrical contact system, the lead body 440 includes regions of insulator material 470 and regions of conductor material 475 (see, e.g., FIG. 4A) that define a stacked rigid region configured to be removably inserted into the bore 465 of the lead connector 445 such that the conductor material 475 is in contact with the matching conductor material 460 of the lead connector 445. The contact between the conductor material 475 and the matching conductor material 460 allows for the conductive material of the one or more leads in the lead body 440 to be in electrical contact with the conductor material 460, and thus allows for electrical coupling of the electronics module 425 to the one or more electrodes 450 via the lead connector 445, flexible cable 447, and feedthrough assembly 420. The conductor material 475 is connected to each respective lead of the lead body 440 via a conductive wire, pad, or tab. The insulator material 470 may be silicone and have a diameter of at least 1 mm, for example from 1.2 mm to 6 mm. The conductor material 475 may be formed of a conductive metal such as a copper, silver, gold, platinum, stainless steel, nickel-cobalt base alloy, platinum-iridium alloy, brass, bronze, aluminum, etc., and have a diameter of at least 1 mm, for example from 1.2 mm to 6 mm.

The flexible cable 447 (e.g., an extension cable) includes one or more leads of conductive material and insulator. The one or more leads carry electrical conductors that allow electrical coupling of the electronics module 425 to the one or more electrodes 450 via the lead connector 445, the lead body 440, and feedthrough assembly 420. In some examples the one or more leads are extruded with a dielectric material such as a polymer having suitable dielectric, flexibility and biocompatibility characteristics. Polyurethane, polycarbonate, silicone, polyethylene, fluoropolymer and/or other medical polymers, copolymers and combinations or blends can be used. The conductive material may be any suitable conductor such as stainless steel, silver, copper or other conductive materials, which may have separate coatings or sheathing for anticorrosive, insulative and/or protective reasons. The conductive material may take various forms including wires, drawn filled tubes, helical coiled conductors, microwires, and/or printed circuits, for example. The feedthrough assembly 420 and the flexible cable 447 may be designed to connect with one another (e.g., via a pin and sleeve connector, snap and lock connector, flexible printed circuit connectors, or other connection techniques). Further, feedthrough assembly 420 and the flexible cable 447 may be overmolded with non-conductive epoxy or silicone to provide mechanical strain relief and electrical isolation. The mechanical strain relief may provide protection from inadvertent bending during implantation, or for bending while implanted in the patient. Further, the strain relief may also provide protection from tension on the feedthrough assembly 420 and the flexible cable 447.

III. Methods for Bonding a Flexible Printed Circuit to a Feedthrough

FIGS. 5-8 depict simplified flowcharts depicting processing performed for bonding a flexible printed circuit to a feedthrough according to embodiments of the present invention. As noted herein, the flowcharts of FIGS. 5-8 illustrate the architecture, functionality, and operation of possible implementations of systems, methods, and computer program products according to various embodiments of the present invention. In this regard, each block in the flowchart or block diagrams may represent a module, segment, or portion of code, which comprises one or more executable instructions for implementing the specified logical functions. It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combination of blocks in the block diagrams and/or flowchart illustration, can be implemented by special purpose hardware-based systems that perform the specified functions or acts, or combinations of special purpose hardware and computer instructions.

FIG. 5 depicts a simplified flowchart 500 illustrating a process for bonding a flexible printed circuit to a feedthrough. In accordance with certain aspects of the present invention, the feedthrough may traverse a housing or cap of a neuromodulator device or system as described herein. At step 505, one or more holes are formed in a substrate of a flexible printed circuit. In various embodiments, an initial wiring level is provided or formed on a surface of the substrate, and photoresist and/or hardmask is deposited on the initial wiring level. Patterned openings are formed in photoresist and/or hardmask selective to the initial wiring level utilizing an etching/removal technique that includes, but is not limited to, dry etching, plasma etching, or reactive ion etching (ME). The initial wiring level may include dielectric layers and multiple metallization layers, wherein the metallization layers can provide electrical connections between components such as the feedthrough and electrodes. The one or more holes may be formed in the initial wiring level and substrate utilizing an etching/removal technique that includes, but is not limited to, ME. For example, the one or more holes can be created by performing anisotropic reactive ion etch of the initial wiring level and substrate. As should be understood, the number of holes and pattern of the holes is selected based on the number and pattern of the feedthroughs or conductive pins of a feedthrough assembly that is being connected to the flexible printed circuit (e.g., a grid pattern).

At step 510, one or more metal annuli are formed on the substrate. In some embodiments, the forming includes patterning each metal annulus around a respective hole formed in step 505 and in contact with a conductive material of the flexible printed circuit (i.e., the initial wiring level). For example, a photoresist and/or hardmask may be deposited on the initial wiring level. A pattern around a respective hole formed in step 505 and in contact with a conductive material of the flexible printed circuit may be formed in photoresist and/or hardmask selective to the initial wiring level utilizing an etching/removal technique that includes, but is not limited to, dry etching, plasma etching, or ME. A metal is then deposited within the pattern using conventional deposition processes. The metal may be, for example, any conductor materials including one or more of titanium, titanium nitride, tungsten, molybdenum aluminum, aluminum-copper, and similar types of materials known to those of skill in the art.

At step 515, a feedthrough assembly having one or more feedthroughs is aligned with the one or more opening formed in step 505. At step 520, the one or more feedthroughs of the feedthrough assembly are placed through the respective one or more holes. At step 525, the one or more feedthroughs are joined to the respective one or more metal annuli. In some embodiments, the joining includes dispensing a conductive epoxy between each feedthrough and the metal annulus, and curing the conductive epoxy. The curing may be performed with heat or ultraviolet light. In alternative embodiments, the joining includes bending in a controlled manner each feedthrough into physical contact with the metal annulus, and attaching each feedthrough to the metal annulus such that the feedthrough is in electrical contact with the metal annulus. The attaching may include dispensing a conductive epoxy between each feedthrough and the metal annulus, and curing the conductive epoxy. Alternatively, the attaching may include laser welding, resistance welding, thermo-compression bonding, ultrasonic bonding, or thermosonic bonding each feedthrough to the metal annulus.

At optional step 530, a region surrounding each feedthrough and metal annulus may be overmolded with non-conductive epoxy or silicone to provide mechanical strain relief and electrical isolation. In some embodiments, the mechanical strain relief may be achieved using mechanical design, for example, as a tapered geometry that gradually increases or decreases from a dimension (e.g., diameter) of the neurostimulator or feedthrough apparatus to a dimension (e.g., diameter) of the flexible printed circuit or flexible cable. The mechanical strain relief may provide protection from inadvertent bending during implantation, or for bending while implanted in the patient. Further, the strain relief may also provide protection from tension on the one or more feedthroughs and/or flexible printed circuit.

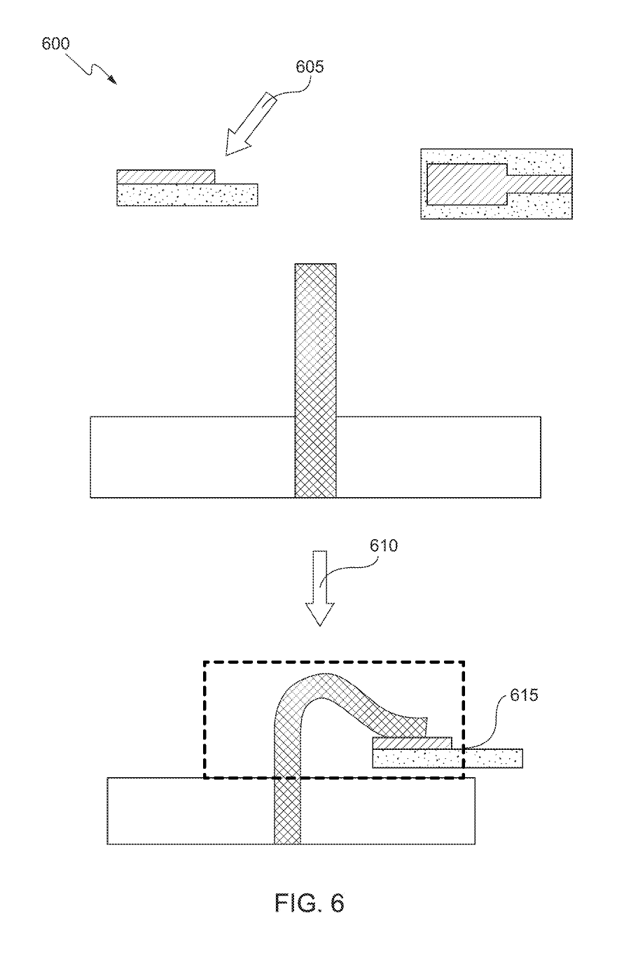

FIG. 6 depicts a simplified flowchart 600 illustrating a process for bonding a flexible printed circuit to a feedthrough. In accordance with certain aspects of the present invention, the feedthrough may traverse a housing or cap of a neuromodulator device or system as described herein. At step 605, one or more bond pads are formed on a substrate of a flexible printed circuit. In some embodiments, the forming includes patterning each bond pad to be in contact with a conductive material of the flexible printed circuit (i.e., the initial wiring level). For example, a photoresist and/or hardmask may be deposited on the initial wiring level. A pattern connecting a bond pad to a conductive material of the flexible printed circuit may be formed in the photoresist and/or hardmask selective to the initial wiring level utilizing an etching/removal technique that includes, but is not limited to, dry etching, plasma etching, or RIE. A metal is then deposited within the pattern using conventional deposition processes. The metal may be, for example, any conductor materials including one or more of titanium, titanium nitride, tungsten, molybdenum aluminum, aluminum-copper, and similar types of materials know to those of skill in the art. In certain embodiments, the bond bad is patterned near an edge of the substrate.

At step 610, one or more feedthroughs are joined to the respective one or more bond pads. In some embodiments, the joining includes aligning to a single feedthrough or linear row of feedthroughs to an edge of the substrate having the one or more bond pads. Thereafter, each feedthrough may be bent in a controlled manner into physical contact with the bond pad, and attaching each feedthrough to the bond pad such that the feedthrough is in electrical contact with the bond pad. The joining or attaching may include dispensing a conductive epoxy between each feedthrough and the bond pad, and curing the conductive epoxy. Alternatively, the joining or attaching may include laser welding, resistance welding, thermo-compression bonding, ultrasonic bonding, or thermosonic bonding each feedthrough and the bond pad.

At optional step 615, a region surrounding each feedthrough and bond pad may be overmolded with non-conductive epoxy or silicone to provide mechanical strain relief and electrical isolation. In some embodiments, the mechanical strain relief may be achieved using mechanical design, for example, as a tapered geometry that gradually increases or decreases from a dimension (e.g., diameter) of the neurostimulator or feedthrough apparatus to a dimension (e.g., diameter) of the flexible printed circuit or flexible cable. The mechanical strain relief may provide protection from inadvertent bending during implantation, or for bending while implanted in the patient. Further, the strain relief may also provide protection from tension on the one or more feedthroughs and/or flexible printed circuit.

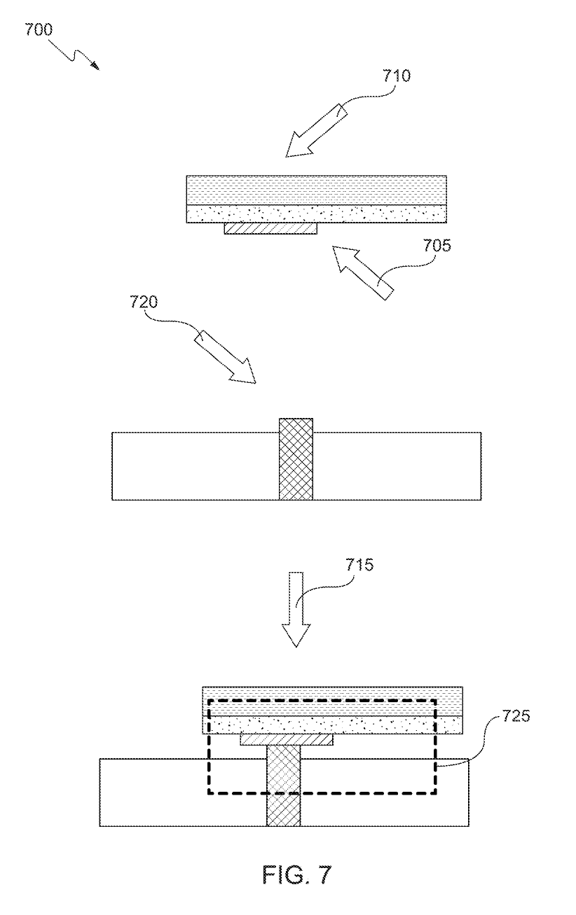

FIG. 7 depicts a simplified flowchart 700 illustrating a process for bonding a flexible printed circuit to a feedthrough. In accordance with certain aspects of the present invention, the feedthrough may traverse a housing or cap of a neuromodulator device or system as described herein. At step 705, one or more bond pads are formed on a substrate of a flexible printed circuit. In some embodiments, the forming includes patterning each bond pad to be in contact with a conductive material of the flexible printed circuit (i.e., the initial wiring level). For example, a photoresist and/or hardmask may be deposited on the initial wiring level. A pattern connecting a bond pad to a conductive material of the flexible printed circuit may be formed in the photoresist and/or hardmask selective to the initial wiring level utilizing an etching/removal technique that includes, but is not limited to, dry etching, plasma etching, or RIE. A metal is then deposited within the pattern using conventional deposition processes. The metal may be, for example, any conductor materials including one or more of titanium, titanium nitride, tungsten, molybdenum aluminum, aluminum-copper, and similar types of materials know to those of skill in the art. In certain embodiments, the bond bad is patterned near an edge of the substrate.

At step 710, a localized backer or stiffener is affixed to the substrate. In some embodiments, the backer or stiffener is affixed in only a region near the one or more bond pads. For example, the backer is on a first side of the substrate opposite to that of the outer planar surface of a feedthrough and the bond pad is on a second side of the substrate adjacent to the outer planar surface of a feedthrough. At step 715, one or more feedthroughs are joined to the respective one or more bond pads. In some embodiments, the joining includes attaching each feedthrough to the bond pad such that each feedthrough is in electrical contact with the bond pad. The attaching may include when the feedthrough is a conductive pin or tab, trimming the feedthrough at step 720 such that the feedthrough protrudes to a height of <2 mm from an outer planar surface of the feedthrough assembly, and welding or thermo-bonding the feedthrough to the bond pad. Alternatively, planar bond pads may be deposited via stencil printing to create a stand-off.

At optional step 725, a region surrounding each feedthrough and bond pad may be overmolded with non-conductive epoxy or silicone to provide mechanical strain relief and electrical isolation. In some embodiments, the mechanical strain relief may be achieved using mechanical design, for example, as a tapered geometry that gradually increases or decreases from a dimension (e.g., diameter) of the neurostimulator or feedthrough apparatus to a dimension (e.g., diameter) of the flexible printed circuit or flexible cable. The mechanical strain relief may provide protection from inadvertent bending during implantation, or for bending while implanted in the patient. Further, the strain relief may also provide protection from tension on the one or more feedthroughs and/or flexible printed circuit.

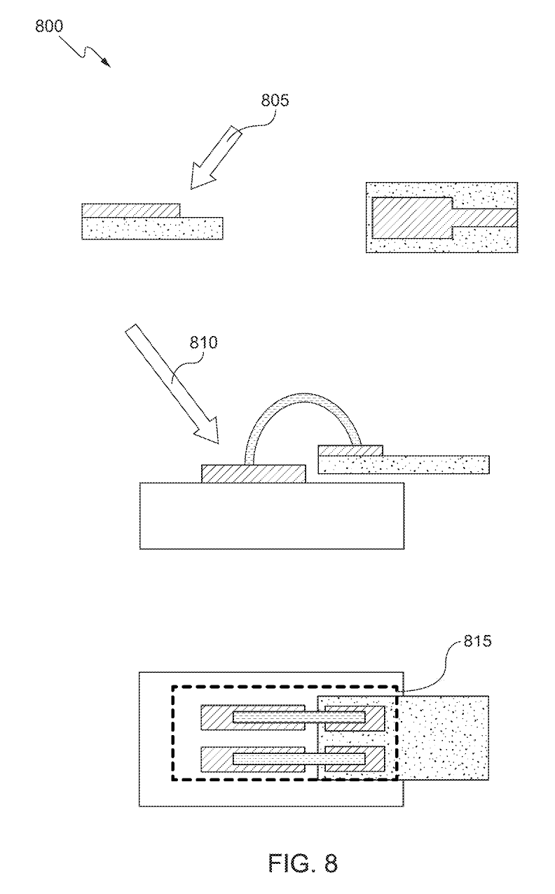

FIG. 8 depicts a simplified flowchart 800 illustrating a process for bonding a flexible printed circuit to a feedthrough. In accordance with certain aspects of the present invention, the feedthrough may traverse a housing or cap of a neuromodulator device or system as described herein. At step 805, one or more bond pads are formed on a substrate of a flexible printed circuit. In some embodiments, the forming includes patterning each bond pad to be in contact with a conductive material of the flexible printed circuit (i.e., the initial wiring level). For example, a photoresist and/or hardmask may be deposited on the initial wiring level. A pattern connecting a bond pad to a conductive material of the flexible printed circuit may be formed in the photoresist and/or hardmask selective to the initial wiring level utilizing an etching/removal technique that includes, but is not limited to, dry etching, plasma etching, or ME. A metal is then deposited within the pattern using conventional deposition processes. The metal may be, for example, any conductor materials including one or more of titanium, titanium nitride, tungsten, molybdenum aluminum, aluminum-copper, and similar types of materials know to those of skill in the art. In certain embodiments, the bond bad is patterned near an edge of the substrate.

At step 810, one or more feedthroughs are joined to the respective one or more bond pads. In some embodiments, the joining includes attaching each feedthrough to the bond pad such that each feedthrough is in electrical contact with the bond pad. The attaching may include when the feedthrough is a conductive pad, bonding a first end of a wire or tab to the feedthrough and bonding a second end of the wire or tab to the bond pad. In some embodiments, a pre-form tab may be interconnected with a bridge to simplify alignment and bonding. After bonding, the bridge elements may be trimmed with a laser.

At optional step 815, a region surrounding each feedthrough and bond pad may be overmolded with non-conductive epoxy or silicone to provide mechanical strain relief and electrical isolation. In some embodiments, the mechanical strain relief may be achieved using mechanical design, for example, as a tapered geometry that gradually increases or decreases from a dimension (e.g., diameter) of the neurostimulator or feedthrough apparatus to a dimension (e.g., diameter) of the flexible printed circuit or flexible cable. The mechanical strain relief may provide protection from inadvertent bending during implantation, or for bending while implanted in the patient. Further, the strain relief may also provide protection from tension on the one or more feedthroughs and/or flexible printed circuit.

IV. Methods of Implanting Neuromodulation Devices or Systems and Treating Rheumatoid Arthritis

Rheumatoid arthritis is a chronic inflammatory disease characterized by synovial inflammation in the musculoskeletal joints resulting in cartilage degradation and bone destruction with consequent disability. Conventional pharmaceutical therapies include glucocorticoids, methotrexate, monoclonal antibodies, and other pharmacological agents targeting inflammatory mechanisms. Despite these treatment options, many rheumatoid arthritis patients fail to respond, instead persisting with poor health, shortened life span, and significant impairments in quality of life. Thus, there remains a significant need for alternative therapeutic approaches such as neuromodulation therapy.

Recent advances in neuroscience have revealed reflex neural circuit mechanisms that regulate innate and adaptive immunity. One such reflex circuit, is the inflammatory reflex, which is defined by signals that travel in the vagus nerve to inhibit the production of tumor necrosis factor and other cytokines. Electrical stimulation of the vagus nerve has been shown to stimulate certain cells to secrete acetylcholine in the spleen and other tissues of animals. Binding of acetylcholine inhibits the nuclear translocation of the NF-κB protein complex and inhibits inflammasome activation in macrophages. Accordingly, inflammatory reflex signaling, which may be enhanced by electrically stimulating the vagus nerve using neuromodulation therapy, has shown to significantly reduce cytokine production by inhibiting inflammasome activation and attenuate the severity of inflammation in some patients with rheumatoid arthritis.

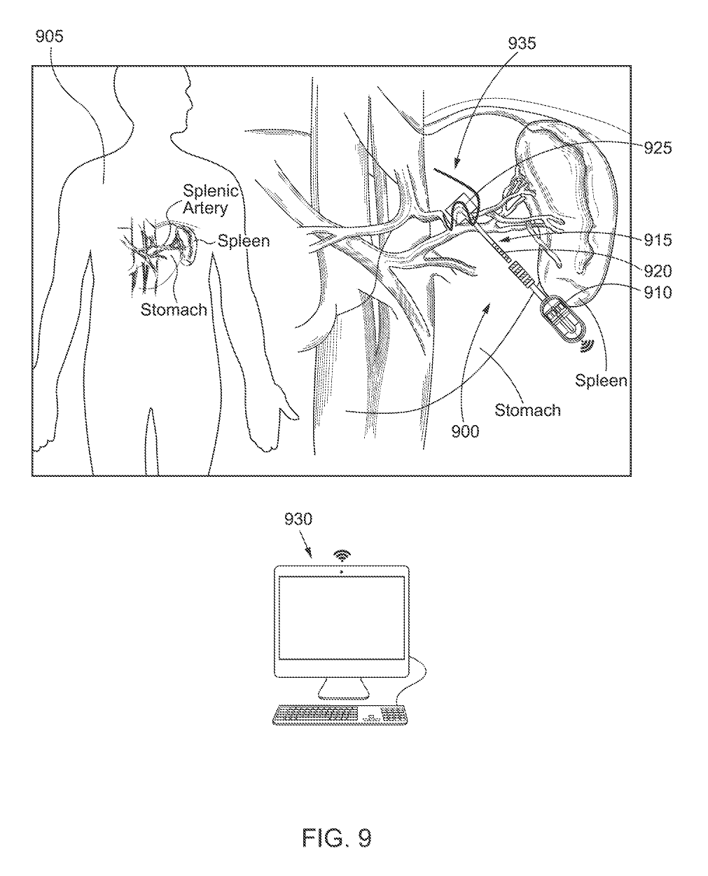

An implantable module neuromodulation device or system may be designed in accordance with various aspects discussed herein for use in treating inflammatory related diseases such as rheumatoid arthritis through therapeutic peripheral nervous system (PNS) stimulation (e.g., stimulation of the vagus nerve). FIG. 9 is a front anatomical diagram showing, by way of example, placement of a neuromodulation device or system 900 (as described with respect to FIGS. 1A-8) in a patient 905. The PNS stimulation provided through the neuromodulation device or system 900 operates under one or more mechanisms of action to treat inflammatory related diseases. The one or more mechanisms may include increasing secretion of acetylcholine and inhibiting inflammation by inhibiting inflammasome activation in macrophages activated by exposure to lipopolysaccharide, other Toll-like receptor ligands, and other proinflammatory stimulating factors.

In some embodiments, the neuromodulation device or system 900 includes at least two implantable components, (i) a neurostimulator 910 comprising a housing, a feedthrough assembly, an electronics module, a power source, and an antenna, and (ii) a lead assembly 915 comprising a lead connector, a lead body 920, and one or more electrodes 925. The one or more electrodes 925 may be provided in a variety of forms, such as, e.g., helical electrodes, probe electrodes, cuff electrodes, as well as other types of electrodes. The neuromodulation device or system 900 may be remotely accessed following implant through an external computing system 930, as seen in FIG. 9, by which the neurostimulator 910 can be remotely controlled, checked, and/or programmed by healthcare professionals and/or the patient. In some embodiments, an electromagnetic controller may enable the patient or healthcare professional to exercise control over therapy delivery and suspension. In other embodiments, an external controller may communicate with the neurostimulator 910 via wired or wireless communication methods, such as, e.g., wireless RF transmission to enable the patient or healthcare professional to exercise control over therapy delivery and suspension. Together, the neuromodulation device or system 900 and external computing system 930 form a PNS therapeutic delivery system.

The neuromodulation device or system 900 may be implanted in the patient's abdominal region on the same side as the nerve or artery/nerve plexus 935 to be stimulated such as the peripheral nerves near the splenic artery or the splenic artery/nerve plexus, although other implantation sites are possible. In various embodiments, the one or more electrodes 925 are connected to the vagus nerve along the splenic artery where access is greatest with the least amount of interference with the stomach, spleen, and pancreas. The size and modularity of the neuromodulation device or system 900 (e.g., a housing having a width of less than 10 mm and a height of less than 10 mm) enables access to the nerve or artery/nerve plexus 935 through a trocar and a laparoscopic procedure. In some embodiments, the neuromodulation device or system 900 includes the neurostimulator 910 placed subdurally at a location that is either remote from or at the neural interface, and removably connected to the lead assembly 915. In alternative embodiments, the neuromodulation device or system 900 includes the neurostimulator 910 placed subdurally at a location that is either remote from or at the neural interface, and permanently attached to the lead assembly 915.

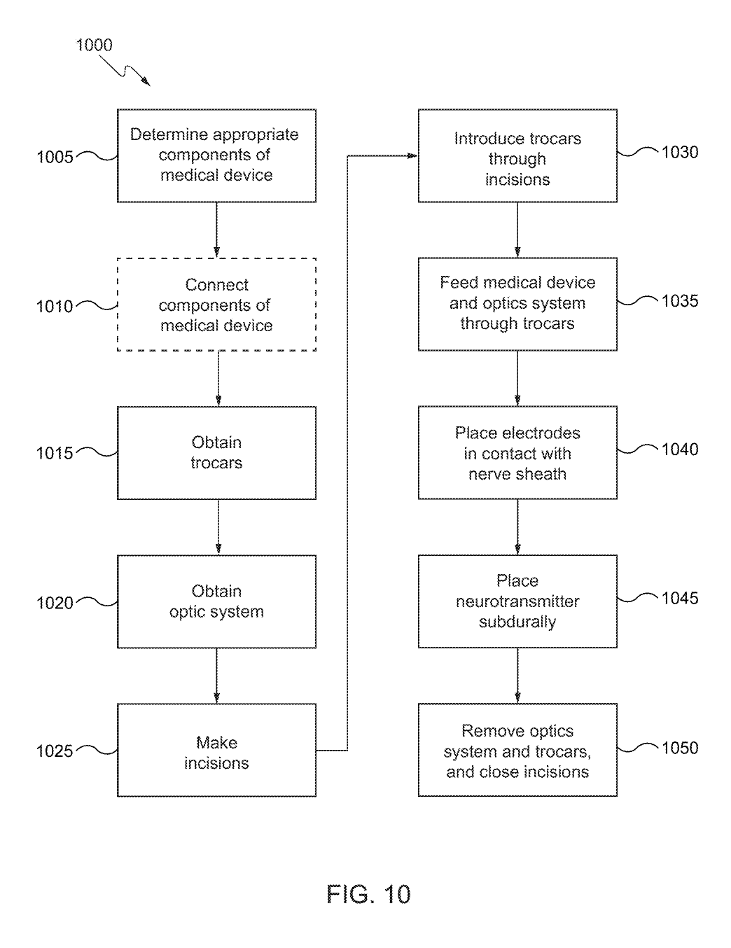

FIGS. 10-12 depict simplified flowcharts depicting processing performed for implanting neuromodulation devices or systems and treating inflammatory related diseases such as rheumatoid arthritis according to embodiments of the present invention. As noted herein, the flowcharts of FIGS. 10-12 illustrate the architecture, functionality, and operation of possible implementations of systems, methods, and computer program products according to various embodiments of the present invention. In this regard, each block in the flowchart or block diagrams may represent a module, segment, or portion of code, which comprises one or more executable instructions for implementing the specified logical functions. It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combination of blocks in the block diagrams and/or flowchart illustration, can be implemented by special purpose hardware-based systems that perform the specified functions or acts, or combinations of special purpose hardware and computer instructions.

FIG. 10 depicts a simplified flowchart 1000 illustrating a process for implanting a neuromodulation device or system. At step 1005, an appropriate neural stimulator and electrode shape and design are determined for a specific patient. For example, a healthcare professional may determine an appropriate neural stimulator and electrode shape and design for a specific patient based on one or more factors such as the anatomical size of the patient's nerve or artery/nerve plexus, the type and/or degree of inflammation occurring within the patient, various stimulation and/or recording requirements to implement a treatment protocol, etc. Optionally at step 1010, in embodiments in which the neurostimulator is removably connectable to the lead assembly, the neuromodulation device or system may be assembled as a single assembly by connecting the determined neurostimulator and lead assembly using a lead connector. For example, in embodiments in which the lead connector is an integrated seal and electrical contact system, the lead body includes regions of insulator material and regions of conductor material (see, e.g., FIG. 2B) at a proximal end that define a stacked rigid region configured to be removably inserted into the bore of the lead connector such that the conductor material is in contact with the matching conductor material of the lead connector. In alternative embodiments in which the neurostimulator is permanently attached to the lead assembly, once the appropriate neural stimulator and electrode are determined, the implantable module neuromodulation device or system may not need to be assembled or connected to construct a single assembly.

At step 1015, one or more trocars are obtained or provided for gaining access to a body cavity (e.g., the abdominal cavity) having a predetermined implantation site (e.g., the vagus nerve along the splenic artery). In various embodiments, each trocar has a diameter of less than 20.0 mm, less than 15.0 mm, or less than 12.0 mm. At step 1020, an optics system is obtained or provided for obtaining images of the implantation site. In various embodiments, the optics system includes a laparoscope having a thin tube with a high-intensity light and a high-resolution camera at the distal end. At step 1025, one or more incisions are made in the patient. In various embodiments, the size of each incision ranges from 0.1 to 2.5 cm, from 0.5 to 1.5 cm, or from 0.5 to 1.0 cm such that the implantation procedure can be maintained as minimally invasive as possible. For example, a scalpel may be used to make one or more incisions having a size from 0.5 to 1.5 cm in the epidermis and dermis of the patient's abdominal region such that the neuromodulation device or system may be implanted in the patient's right or left abdominal region on the same side as the vagus nerve to be stimulated, although other incision and implantation sites, are possible.

At step 1030, the one or more trocars are introduced through the one or more incisions and used to gain access to the body cavity. In various embodiments, the one or more trocars may be introduced through the one or more incisions and passed through one or more layers of dermis, epidermis, fascia, peritoneum, fat, muscle, etc. to gain access to the body cavity. Once access to the body cavity is obtained, the one or more trocars may be used to introduce various instruments and material into the body cavity such as inflating the cavity with gas via a gas intake port. At step 1035, the optics system and the neuromodulation device or system are fed through the one or more trocars to the implantation site. In some embodiments, the neuromodulation device or system is fed through a trocar to the implantation site for connecting the one or more electrodes to the nerve or artery/nerve plexus, and the optics system is fed through another trocar to the implantation site for obtaining images of the neuromodulation device or system at the implantation site to assist in connecting the one or more electrodes to the nerve or artery/nerve plexus.

At step 1040, the one or more electrodes are placed into contact with the exposed nerve sheath at the implantation site and optionally tethered. For example, the one or more electrodes may be placed into contact with a nerve or artery/nerve plexus such as the peripheral nerves near the splenic artery or the splenic artery/nerve plexus. The contact creates a neural interface between the one or more electrodes and the exposed nerve sheath that allows for stimulation (e.g., electrical stimulation) to be passed from the one or more electrodes to the nerve or artery/nerve plexus. At step 1045, the neurotransmitter is placed subdurally at a location that is either remote from or at the neural interface, and optionally tethered. Placed subdurally at a location that is “remote” means that the neurotransmitter is placed in a location subdurally that is greater than 5 cm, 10 cm, or 20 cm from the one or more electrodes. Placed subdurally at a location that is “at” the neural interface means that the neurotransmitter is placed in a location subdurally that is less than 5 cm, 3 cm, or 1 cm from the one or more electrodes. For example, a subcutaneous tunnel maybe formed between the implantation site of the one or more electrodes, through which the lead connector and/or the lead body is guided, and the neurotransmitter may be placed subdurally at a location remote from the one or more electrode. Alternatively, the neurotransmitter may be placed subdurally at the neural interface between the one or more electrodes and the exposed nerve sheath. At step 1050, the optics system and the one or more trocars are removed, the one or more incisions are closed with stitches or surgical tape, and the wounds are bandaged.

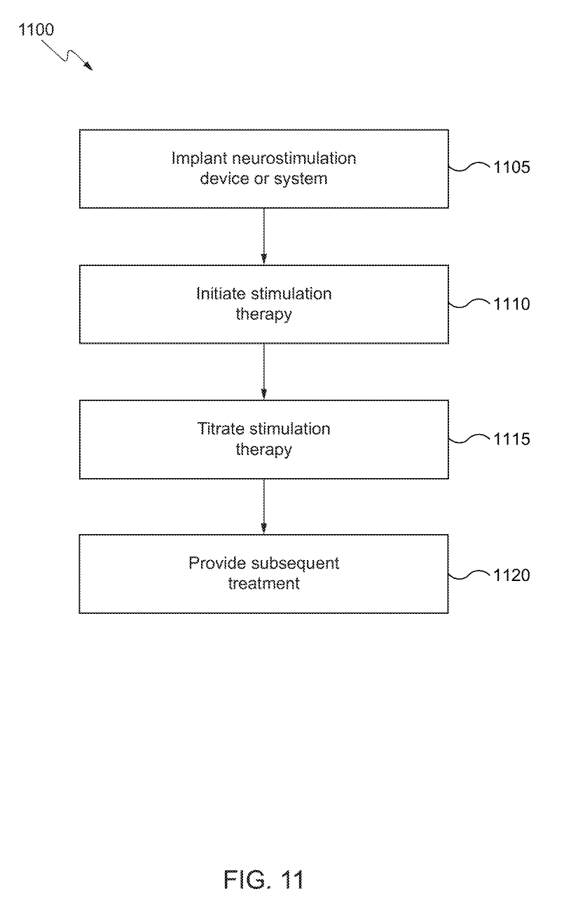

FIG. 11 depicts a simplified flowchart 1100 illustrating a titration process used to gradually increase stimulation intensity (i.e., a combination of one or more of the stimulation parameters) to a desired therapeutic level for treating inflammatory related diseases such as rheumatoid arthritis. At step 1105, a neuromodulation device or system is implanted subcutaneously in a patient. In various embodiments, the neuromodulation device or system includes at least (i) a neurostimulator including a housing having a width of less than 10 mm and a height of less than 10 mm, and (ii) a lead assembly including one or more electrodes connected to the neurostimulator, as described with respect to FIGS. 1A-9. The neuromodulation device or system may be implanted as described with respect to FIG. 10. For example, during the implanting, the one or more electrodes are connected to a nerve or artery/nerve plexus such as the peripheral nerves near the splenic artery or the splenic artery/nerve plexus. The neurostimulator is positioned subdurally or at the neural interface. The one or more electrodes and the neurostimulator may be connected via one or more leads of the lead assembly and lead connector either prior to implanting in step 1105 or after positioning of the one or more electrodes in proximity to the nerve or artery/nerve plexus.

In step 1110, a stimulation therapy process is initiated. In some embodiments, the stimulation therapy is initiated after an optional post-surgery recovery period (e.g., a number of days/weeks), during which time no stimulation therapy occurs. In alternative embodiments, the stimulation therapy is initiated shortly (e.g., hours) or immediately after implantation. Initiation of the stimulation therapy may include obtaining or generating an initial set of stimulation parameters and providing stimulation to the patient using the initial set of stimulation parameters. The initial set of stimulation parameters may comprise one or more of an initial burst duration, initial burst interval, initial stimulation amplitude, initial pulse width, initial frequency, initial duty cycle, initial stimulation waveform shape, and initial electrode configuration. The various initial parameter settings may vary, but may be selected so that one or more of the parameters are set at levels below a predefined target parameter set level, such that the titration process is used to gradually increase the intensity parameters to achieve adequate adaptation. In some embodiments, the initial burst duration, the initial burst interval, the initial frequency, and initial electrode configuration are set at target levels or configurations, while the initial stimulation amplitude, initial pulse width, stimulation waveform shape, and initial duty cycle are set below their respective target levels. In other embodiments, the initial electrode configuration is set at a target level or configuration, while the initial burst duration, the initial burst interval, the initial frequency, the initial stimulation amplitude, the initial pulse width, stimulation waveform shape, and the initial duty cycle are set below their respective target levels.

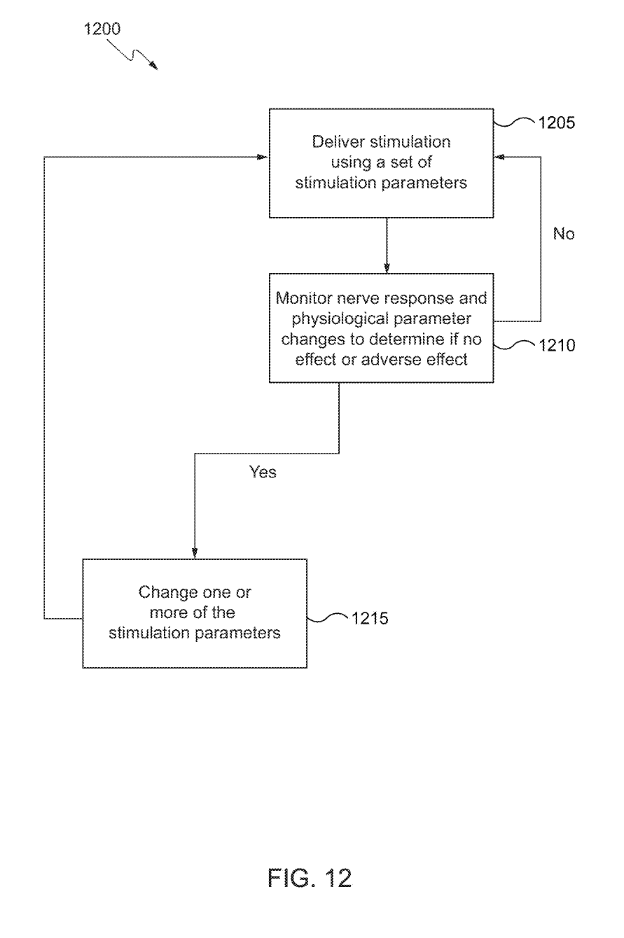

In step 1115, stimulation therapy is provided using the initial set of stimulation parameters and titrated by setting or adjusting the stimulation parameters using a titration schedule to obtain or generate subsequent sets of stimulation parameters with the goal of achieving adequate adaptation. In some embodiments, the titration process includes delivering stimulation using a neurostimulator based on a set of stimulation parameters, monitoring a response to the stimulation that includes monitoring of nerve responses, physiological parameter changes (such as changes in inflammation, blood pressure, perceived pain, etc.), or a combination thereof, modifying one or more of the stimulation parameters based on a titration schedule, the nerve responses, and/or the physiological changes to create a subsequent set of stimulation parameters, and delivering the neural stimulation using the neurostimulator based on the subsequent set of stimulation parameters. This process may be repeated until adequate adaptation is achieved. In some embodiments, adequate adaptation includes achieving a target intensity level for one or more stimulation parameters and/or a desired physiological effect. The achievement of one or more of these objectives determines the stimulation intensity including a therapeutic set of stimulation parameters to be used for subsequent treatment doses delivered during the remainder of stimulation therapy in step 1120, as further described herein with respect to FIG. 12.

FIG. 12 depicts a simplified flowchart 1200 illustrating a process used to provide a therapeutic level of neural stimulation for treating inflammatory related diseases such as rheumatoid arthritis. In step 1205, the neuromodulation system delivers stimulation to the patient using a set of stimulation parameters. In some embodiments, the set of stimulation parameters are the set of stimulation parameters determined in step 1115 of FIG. 11, that has achieved adequate adaptation. In step 1210, the nerve responses and physiological parameter changes are monitored to determine whether the stimulation provided using the set of stimulation parameters achieved a desired physiological effect (e.g., decreased inflammation), had no physiological effect (e.g., no change in the level of inflammation), and/or had an adverse physiological effect on the patient (e.g., increased inflammation). In some embodiments, prior to neural stimulation, baseline values for biomarkers of inflammation may be determined and recorded for a patient. In some embodiments, the baseline values may be recorded in the memory of an external computing system (e.g., the memory of external computing system 930 as described with respect to FIG. 9). Once neural stimulation begins on the patient, the values obtained for biomarkers of inflammation may be compared respectively to the baseline values to determine the extent of change in the physiological parameters such as inflammation. The determined extent of change for the physiological parameters may then be compared to predetermined threshold values set for the physiological parameters to determine whether the stimulation provided using a set of stimulation parameters achieved a desired physiological effect, had no physiological effect, or an adverse physiological effect. When the stimulation provided using a set of stimulation parameters is determined to have had no physiological effect or an adverse physiological effect, the process proceeds to step 1215. When the stimulation provided using a set of stimulation parameters is determined to have achieved a desired physiological effect, the process proceeds to step 1205 and continues to monitor nerve responses and physiological parameter changes through-out the remainder of the stimulation therapy.

In step 1215, one or more of the stimulation parameters is changed to achieve a desired physiological effect and/or minimize or prevent the adverse physiological effect. In some embodiments, one or more of the burst duration, the burst interval, the stimulation amplitude, the pulse width, the frequency, the duty cycle, stimulation waveform shape, and the electrode configuration is changed to achieve a desired physiological effect and/or minimize or prevent the adverse physiological effect. The changes in the parameter settings may vary, but may be selected so that one or more of the parameters are set at levels above or below previously set levels, such that the desired physiological effect is achieved and/or the adverse physiological effect is minimized or prevented from reoccurring during the remainder of stimulation therapy. In some embodiments, the burst duration, the burst interval, the frequency, and electrode configuration are maintained at target levels or configurations, while one or more of the stimulation amplitude, the pulse width, stimulation waveform shape, and the duty cycle are reduced below their respective previous levels. In other embodiments, an initial electrode configuration is maintained at a target level or configuration, while one or more of the burst duration, the burst interval, the frequency, the stimulation amplitude, the pulse width, stimulation waveform shape, and the duty cycle are reduced below their respective previous levels. In yet other embodiments, the burst duration, the burst interval, the frequency, the stimulation amplitude, the pulse width, stimulation waveform shape, and the duty cycle are maintained at target levels, while the electrode configuration is modified. Once the one or more of the stimulation parameters is changed, the process proceeds to step 1205, to deliver stimulation to the patient using the modified set of stimulation parameters and monitor nerve responses and physiological parameter changes to determine whether the stimulation provided using the modified set of stimulation parameters achieved the desired physiological effect and/or minimized or prevented the adverse physiological effect.

While the invention has been described in detail, modifications within the spirit and scope of the invention will be readily apparent to the skilled artisan. It should be understood that aspects of the invention and portions of various embodiments and various features recited above and/or in the appended claims may be combined or interchanged either in whole or in part. In the foregoing descriptions of the various embodiments, those embodiments which refer to another embodiment may be appropriately combined with other embodiments as will be appreciated by the skilled artisan. Furthermore, the skilled artisan will appreciate that the foregoing description is by way of example only, and is not intended to limit the invention.

1. A medical device comprising:
an implantable neurostimulator including:
a housing having a width of less than 10 mm and a height of less than 10 mm;
a cap bonded to the housing;
one or more feedthroughs that pass through the cap; and
an electronics module within the housing and connected to the one or more feedthroughs; and
a lead assembly including:
a lead body including a conductor material;
a lead connector that connects the conductor material to the one or more feedthroughs; and
one or more electrodes connected to the conductor material.

2. (canceled)

3. The medical device of claim 1, wherein the implantable neurostimulator further includes an antenna connected to the electronics module.

4-8. (canceled)

9. The medical device of claim 1, wherein the implantable neurostimulator further includes one or more feedthroughs that pass through a proximal end of the implantable neurostimulator, the one or more feedthroughs that pass through the cap are provided at a distal end of the implantable neurostimulator, and the electronics module is connected to the one or more feedthroughs at the proximal end of the implantable neurostimulator.

10-11. (canceled)

12. The medical device of claim 1, wherein the one or more feedthroughs comprise a ferrule that defines an aperture, a conductive element passing through the aperture, and an insulator within the aperture surrounding the conductive element and being brazed to the ferrule.

13-22. (canceled)

23. The medical device of claim 1, wherein the lead body includes an insulator and the conductor material, and wherein the lead connector includes an insulator and a matching conductor material defining a common bore configured to removably receive the lead body such that the conductor material of the lead body is in contact with the matching conductor material of the lead connector.

24. (canceled)

25. The medical device of claim 1, wherein the leady body is a flexible printed circuit or flexible cable.

26. (canceled)

27. The medical device of claim 1, wherein the lead body includes an insulator and the conductor material, and wherein the lead connector includes an insulator and a matching conductor material defining a common bore configured to receive the lead body such that the conductor material of the lead body is in contact with the matching conductor material of the lead connector.

28-29. (canceled)

30. The medical device of claim 1, wherein the one or more electrodes are helical electrodes, probe electrodes, or cuff electrodes.

31. A medical device comprising:
an implantable neurostimulator including:
a housing having a width of less than 10 mm and a height of less than 10 mm;
one or more feedthroughs that pass through the housing; and
an electronics module within the housing and connected to the one or more feedthroughs; and
a lead assembly including:
a lead body including: a proximal end having an insulator and a conductor material, and one or more leads sheathed in an insulator and connected to the conductor material;
a lead connector including: an insulator and a matching conductor material defining a common bore configured to removably receive the lead body such that the conductor material of the lead body is in contact with the matching conductor material of the lead connector; and
one or more electrodes connected to the one or more leads,
wherein the matching conductor material of the lead connector is connected to the one or more feedthroughs.

32. (canceled)

33. The medical device of claim 31, wherein the implantable neurostimulator further includes an antenna connected to the electronics module.

34-37. (canceled)

38. The medical device of claim 31, wherein the implantable neurostimulator further includes one or more feedthroughs that pass through a proximal end of the implantable neurostimulator, the one or more feedthroughs that pass through the housing are provided at a distal end of the implantable neurostimulator, and the electronics module is connected to the one or more feedthroughs at the proximal end of the implantable neurostimulator.

39-44. (canceled)

45. The medical device of claim 31, wherein the one or more electrodes are helical electrodes, probe electrodes, or cuff electrodes.

46. The medical device of claim 31
wherein the housing is hemispherical; and
wherein the matching conductor material of the lead connector is connected to the one or more feedthroughs via a flexible cable.

47. (canceled)

48. The medical device of claim 46, wherein the implantable neurostimulator further includes an antenna connected to the electronics module.

49. (canceled)

50. The medical device of claim 48, herein the antenna is a planar coil made of flex substrate and a microwire coil.

51-56. (canceled)

57. The medical device of claim 46, wherein the one or more electrodes are helical electrodes, probe electrodes, or cuff electrodes.

58-84. (canceled)

85. A method of treating an inflammatory related disease comprising:
implanting a medical device in a body cavity using a laparoscopic procedure, wherein the medical device comprises: (i) a neurostimulator including a housing having a width of less than 10 mm and a height of less than 10 mm, and (ii) a lead assembly including one or more electrodes connected to the neurostimulator, and the implanting comprises connecting the one or more electrodes to a nerve or artery/nerve plexus in the body cavity;
delivering, by a computing system, neural stimulation to the nerve or artery/nerve plexus based on a first set of stimulation parameters;
monitoring, by the computing system, a response to the neural stimulation that includes monitoring responses of the nerve or artery/nerve plexus and a physiological parameter change;
modifying, by the computing system, the first set of the stimulation parameters based on the responses of the nerve or artery/nerve plexus and the physiological parameter change to create a second set of stimulation parameters; and
delivering, by the computing system, the neural stimulation based on the second set of the stimulation parameters.

86. (canceled)

87. The method of claim 85, wherein the stimulation parameters include at least one of: stimulation amplitude, pulse width, frequency, duty cycle, stimulation waveform shape, and electrode configuration.

88-89. (canceled)

90. The method of claim 85, wherein the monitoring the response to the neural stimulation includes determining whether the neural stimulation has a desired physiological effect on the inflammation of the patient, obtaining values for biomarkers of the inflammation, and comparing the values for the biomarkers to baseline values to determine an extent of change in the inflammation.

91-94. (canceled)

95. The method of claim 85, wherein the method further includes determining whether adequate adaptation is achieved, and wherein adequate adaptation is achieved when at least one of the following objectives is achieved: a target intensity level for one or more of the stimulation parameters and/or a desired physiological effect.

96-97. (canceled)
